(19) United States
(12) Patent Application Publication
(10) Pub. No.: US 2018/0084766 A1
(43) Pub. Date: Mar. 29, 2018

(54) METHODS AND COMPOSITIONS RELATING TO AN EMBRYONIC STEM CELL-BASED TUMOR MODEL

(71) Applicant: THE CHILDREN'S MEDICAL CENTER CORPORATION, Boston, MA (US)

(72) Inventors: Frederick W. Alt, Cambridge, MA (US); Feilong Meng, Brookline, MA (US); Zhaoqing Ba, Boston, MA (US); Monica Gostissa, Jamaica Plain, MA (US); Baochun Zhang, Newton, MA (US); Pei-Chi Wei, Brookline, MA (US); Bjoern Schwer, San Francisco, CA (US)

(73) Assignee: THE CHILDREN'S MEDICAL CENTER CORPORATION, Boston, MA (US)

(21) Appl. No.: 15/564,647

(22) PCT Filed: Apr. 7, 2016
(86) PCT No.: PCT/US16/26404

§ 371 Date: Oct. 5, 2017

Related U.S. Application Data
(60) Provisional application No. 62/144,015, filed on Apr. 7, 2015.

(51) Int. Cl. A01K 67/027; A61K 49/00; C07K 14/82; C07K 14/005; C12N 7/00

(52) U.S. Cl. CPC A01K 67/0275; A61K 49/0008; A01K 67/0271; C07K 14/82; C07K 14/005; C12N 2710/16251; A01K 2207/12; A01K 2227/105; A01K 2217/05; A01K 2267/0331; A01K 2217/15; C12N 7/00

(57) ABSTRACT

Provided herein are rapid, reliable methods for generating mice (or other species) that develop tumors of known genotype with respect to two or more mutated, tumor-associated genes using blastocyst complementation.

### CROSS-REFERENCE TO RELATED APPLICATIONS

This Application claims benefit under 35 U.S.C. § 119(e) of the U.S. Provisional Application No. 62/144,015 filed Apr. 7, 2015, the contents of which are incorporated herein by reference in their entirety.

### FIELD OF THE INVENTION

The field of the invention relates to animal models for cancer and methods for making the same.

### BACKGROUND

Epstein-Barr virus (EBV) is a γ herpes virus that can infect and transform human B lymphocytes. EBV causes a variety of human pathologies, ranging from nonmalignant diseases such as infectious mononucleosis to malignant diseases such as post-transplantation lymphoproliferative disorder (PTLD), AIDS-associated B cell lymphomas, and X-linked lymphoproliferative disorder-associated B cell lymphomas in immuno-compromised hosts (1). EBV can also cause Burkitt and Hodgkin lymphomas in immunocompetent hosts (1). After a B cell is infected, EBV infection drives B cell proliferation, but the infected cells are under tight immune surveillance by T cells and natural killer (NK) cells that develops upon EBV infection and largely eradicates infected B cells (2). Subsequently, EBV infection reaches a latent stage, which can persist in a small fraction of B cells for life. Under immunosuppression, EBV can spread and cause massive B cell expansion and malignant transformation (1). The EBV latent membrane protein 1 (LMP1) is a major contributing factor to the activation and transformation of human B cells (3). LMP1 is a transmembrane protein that functionally mimics a constitutively active B cell CD40 co-receptor, which signals through the tumor necrosis factor (TNF) pathway to activate downstream NF-κB, ERK, JNK, and JAK/STAT signaling pathways that promote cell growth and survival (4-6).

Indeed, ectopic LMP1 expression can transform rodent fibroblasts (7). A mouse model to study LMP1-induced immune surveillance and lymphoma has been developed based on the conditional expression of LMP1 in mouse B cells using B cell-specific CD19-driven Cre/loxP-mediated recombination to activate LMP1 expression (8,9). In such “CD19-cre; LMP1stopFL/+” mice, LMP1+ B cells were eliminated by T cell immune responses leading to a reduction in the number of splenic B cells compared to that of WT mice, a phenomenon reminiscent of the clearance of EBV-infected human B cells by the host immune system (2). However, elimination of the host T cell immune response by crossing into backgrounds with homozygous elimination of both TCRα/β and TCRγ/δ T cells (TCRβ−/−δ−/−) resulted in rapid fatal LMP1+ B cell expansions and B cell lymphomas in the compound mutant mice (subsequently referred to as “CLT” mice) (8). Thus, elimination of T cell immune surveillance in mice allows ectopic LMP1 expression in mouse B cells to routinely cause robust B cell proliferation and aggressive B cell lymphoma (8). Notably, the LMP1+lymphomas that develop in CLT mice routinely express Activation-Induced Cytidine Deaminase (AID) (8), which is consistent with findings that AID expression is up-regulated by LMP1 (10,11). AID initiates mutations during immunoglobulin (Ig) gene variable region somatic hypermutation process and DNA double stranded breaks during the Ig heavy (IgH) class switch recombination process (12,13). Off-target AID activity has been implicated in certain B cell lymphomas in mice and humans, including human Burkitt lymphoma. On this basis a role of AID expression in CLT B cell lymphomagenesis (8), and other B cell lymphomagenesis (13-16) has been considered.

The CLT mouse tumor model is but one model for which it is difficult, if not impossible, to keep breeding stocks of mice that will faithfully and efficiently reproduce the same tumor genotype.

### SUMMARY

The methods and compositions provided herein are based, in part, on the discovery that a transgenic mouse tumor model can be generated rapidly and efficiently using blastocyst complementation. Proof of principle is provided by the efficient generation of mice with a complex lymphoma genotype using cells derived from the CLT mouse for blastocyst complementation. The methods described herein provide a source of tumor model animals, each bearing tumors of the same genotype, in an approach that does not require animal cross-breeding or the maintenance of extensive parental animal stocks.

Provided herein, in one aspect, is a method for generating a complex mouse tumor model, the method comprising: (a) introducing an embryonic stem (ES) cell or induced pluripotent stem (iPS) cell derived from a complex mouse tumor model and having a modified genome to a donor blastocyst to produce a chimeric blastocyst, and (b) implanting the chimeric blastocyst into a female mouse for gestation, thereby generating a complex mouse tumor model.

In one embodiment of this aspect and all other aspects described herein, the complex mouse tumor model is homozygous recessive for one or more tumor-related genes.

In another embodiment of this aspect and all other aspects described herein, the modified genome comprises at least one insertion, deletion, or mutation.

In another embodiment of this aspect and all other aspects described herein, the genome modification comprises addition of an activated oncogene.

In one embodiment of this aspect and all other aspects described herein, the method provides the mice from the complex tumor mouse model faster than conventional back-crossing methods.

Also provided herein, in another aspect, is a complex mouse tumor model made by the methods described herein.

Provided herein, in another aspect, is a method for generating a transgenic mouse lymphoma model, the method comprising: (a) introducing an embryonic stem (ES) cell or induced pluripotent stem (iPS) cell comprising a modified genome to a RAG-2-deficient blastocyst to produce a chimeric blastocyst, and (b) implanting the chimeric blastocyst into a female mouse for gestation, thereby generating a transgenic mouse lymphoma model.

In one embodiment of this aspect and all other aspects described herein, the ES cell or iPS cell is derived from a tumor model mouse.

In another embodiment of this aspect and all other aspects described herein, the tumor model mouse is homozygous recessive for one or more tumor-related genes.

In another embodiment of this aspect and all other aspects described herein, the modified genome comprises at least one insertion, deletion, or mutation.

In another embodiment of this aspect and all other aspects described herein, the genome modification comprises addition of an activated oncogene or a latent viral gene.

In another embodiment of this aspect and all other aspects described herein, the transgenic mouse comprises chimeric lymphocytes having the genotype of the ES or iPS cell.

In another embodiment of this aspect and all other aspects described herein, at least 50% of the lymphocytes in the transgenic mouse have a genotype derived from the ES or iPS cell.

In another embodiment of this aspect and all other aspects described herein, the latent viral gene is derived from the Epstein-Barr virus.

In another embodiment of this aspect and all other aspects described herein, the latent viral gene is LMP-1.

In another embodiment of this aspect and all other aspects described herein, the transgenic mouse comprises B and/or T cells derived from the ES or iPS cell.

In another embodiment of this aspect and all other aspects described herein, the method provides transgenic mice faster than conventional back-crossing methods.

A transgenic mouse lymphoma model made by any of the methods described herein.

Another aspect provided herein relates to a RAG-2 deficient transgenic mouse lymphoma model, comprising: chimeric lymphocytes having a modified genome, wherein the genotype of the lymphoma is that of the chimeric lymphocytes.

In one embodiment of this aspect and all other aspects described herein, at least 50% of the lymphocytes in the transgenic mouse are chimeric lymphocytes (e.g., at least 55%, at least 60%, at least 65%, at least 70%, at least 75%, at least 80%, at least 85%, at least 90%, at least 95%, at least 98%, at least 99%, or even 100%).

In another embodiment of this aspect and all other aspects described herein, the modified genome comprises at least one insertion, deletion, or mutation.

In another embodiment of this aspect and all other aspects described herein, the modified genome comprises a latent viral gene or an oncogene.

In another embodiment of this aspect and all other aspects described herein, the latent viral gene is derived from the Epstein-Barr virus.

In another embodiment of this aspect and all other aspects described herein, the latent viral gene is LMP-1.

In another embodiment of this aspect and all other aspects described herein, the transgenic mouse further comprises at least one symptom of a B cell expansion or a B cell lymphoma.

In another embodiment of this aspect and all other aspects described herein, the at least one symptom is selected from the group consisting of: splenomegaly, splenic tumor nodules, hepatomegaly, and hepatic tumor nodules.

In another embodiment of this aspect and all other aspects described herein, the B cells comprise at least one cell surface marker profile selected from the group consisting of: CD19⁺, CD95/Fas⁺, B220_{low}, IgM⁺, Igk⁺, CD4⁻ and CD8⁻.

In another embodiment of this aspect and all other aspects described herein, the chimeric lymphocytes are derived from a tumor mouse model.

In another embodiment of this aspect and all other aspects described herein, the tumor mouse model is homozygous recessive for one or more tumor-related genes.

In another embodiment of this aspect and all other aspects described herein, the tumor mouse model comprises a genotype that is TCRβ^{-/-}δ^{-/-}.

In another embodiment of this aspect and all other aspects described herein, the tumor mouse model is the CLT mouse model.

Also provided herein, in another aspect, is a screening assay for identifying an anti-cancer agent for the treatment of lymphoma, the assay comprising: (a) determining the extent of B cell expansion or lymphoma in a RAG-2 deficient transgenic mouse, wherein the RAG-2 deficient transgenic mouse comprises chimeric lymphocytes having a modified genome and further comprises a lymphoma having a genotype of the chimeric lymphocytes, (b) administering a candidate anti-cancer agent to the RAG-2 deficient mouse, and (c) comparing the extent of B cell expansion or lymphoma in the treated RAG-2 deficient mouse, wherein a decrease in the extent of B cell expansion or lymphoma identifies the candidate as an anti-cancer agent for treatment of lymphoma.

In one embodiment of this aspect and all other aspects described herein, the extent of B cell expansion or lymphoma is determined by measuring spleen size.

In another embodiment of this aspect and all other aspects described herein, spleen size is determined using ultrasound technology.

Also provided herein, in another aspect, is a method for generating a medulloblastoma model mouse, the method comprising: (a) introducing a p53^{-/-}, Nestin-Cre/XRCC4-conditional murine embryonic stem cell or induced pluripotent stem cell to a murine blastocyst to produce a chimeric blastocyst; and (b) implanting the chimeric blastocyst into a female mouse for gestation, whereby resulting chimeric p53^{-/-}/Nestin-Cre/XRCC4-conditional mice are predisposed to the development of medulloblastoma.

In one embodiment of this aspect and all other aspects provided herein, resulting chimeric mice are XRCC4⁻ in neuronal tissue.

In another embodiment of this aspect and all other aspects provided herein, the murine blastocyst is from a wildtype animal.

Another aspect provided herein relates to a method for generating a medulloblastoma mouse model, the method comprising: (a) introducing a murine embryonic stem cell or induced pluripotent stem cell to a murine blastocyst to produce a chimeric blastocyst, wherein the embryonic stem cell or induced pluripotent stem cell is p53^{-/-} and carries a Cre-recombinase coding sequence operably linked to a neuronal-tissue-specific promoter and XRCC4 coding sequence flanked by loxP sites; and (b) implanting the chimeric blastocyst into a female mouse for gestation, whereby resulting chimeric p53^{-/-}/Cre/XRCC4-conditional mice are predisposed to the development of medulloblastoma.

In one embodiment of this aspect and all other aspects provided herein, the resulting chimeric mice are XRCC4⁻ in neuronal tissue.

In another embodiment of this aspect and all other aspects provided herein, the murine blastocyst is from a wildtype animal.

Another aspect described herein relates to a medulloblastoma mouse model made by any one of the methods described herein.

Another aspect provided herein relates to use of blastocyst complementation with an ES or iPS cell from a complex tumor model animal to produce an animal with the complex tumor model phenotype.

### BRIEF DESCRIPTION OF THE FIGURES

FIG. 1 Schematic flow chart of generating mouse models of LMP1-driven lymphomas via RAG-2-deficient blastocyst complementation (RDBC). Mice with the indicated genotypes were bred and ES cell lines (CLT) were derived from the embryos. CLT ES cells were genotyped and injected into RAG2-deficient blastocysts to yield chimeric mice in which all peripheral lymphocytes derive from the injected ES cells.

FIGS. 2A-2D CLT RDBC chimeras develop LMP1-driven polyclonal expansions and clonal B-cell lymphomas. FIG. 2A, Kaplan-Meier curves showing survival of mice within a cohort of 21 CLT RDBC chimeras are plotted. FIG. 2B, Representative images of spleen from control and CLT RDBC chimeras. Scale bar, 5 mm. FIG. 2C, Representative FACS analysis of splenic cells from control and CLT RDBC chimeras. The surface markers examined are indicated on the right. FIG. 2D, Southern blotting analysis of EcoRI-digested genomic DNA of samples separated from spleen (S) and liver (L) of the control and CLT RDBC chimeras for rearrangements of the IgH JH region and downstream Cμ and Cδ constant region exons. Top: diagram represents IgH locus with positions of EcoRI sites, the locations of the JH segments, and the position of the JH probe used. Lower: Southern blot panel of DNAs from indicated control and tumor samples from spleen (S), liver (L) and purified splenic B cells (B). Red arrowheads indicate clonal JH rearrangements. Molecular weight (kb) markers and probes are indicated on the left, and the running position of the germline (gl) 6.2 kb JH locus-containing band is indicated on the right.

FIGS. 3A-3D Generation of AID−/− CLT ES cells by a CRISPR/Cas9 designer nuclease approach. FIG. 3A, Schematic diagrams of mouse AID genomic locus and AID knock-out allele with positions of indicated exons, BglII sites, Cas9/gRNA targeting sites, and the probe used for Southern blotting. FIG. 3B, Southern blotting analysis of BglII-digested genomic DNA from parental CLT ES cells, one clone of CLT; AID+/−ES cells, and two independent clones of CLT; AID−/− ES cells. Molecular weight (kb) markers are indicated on the left. FIGS. 3C-3D, Real-time qRT-PCR (FIG. 3C) and Western blotting (FIG. 3D) analyses of AID expression in splenic B-cell population from indicated CLT and CLT; AID−/− RDBC chimeras. The mouse B-cell lymphoma cell line CH12F3 and splenic B cells purified from control mice treated with indicated cytokines were used as the control for AID expression. In FIG. 3C, the AID expression level was normalized to HPRT level. In FIG. 3D, tubulin was detected in parallel and used as a loading control. The asterisk (*) indicates a non-specific cross-reactive band. CIT: α-CD40+IL-4+TNF-β.

FIGS. 4A-4D Comparative analysis of LMP1-driven polyclonal expansions and clonal B-cell lymphomas derived from CLT and CLT; AID−/− RDBC chimeras. FIG. 4A, Kaplan-Meier curves showing survival of mice within cohorts of CLT and CLT; AID−/− RDBC chimeras. Cohorts of 8 CLT RDBC chimeras and 16 CLT; AID−/− RDBC chimeras were plotted. N.s. means statistically not significant by Gehan-Breslow-Wilcoxon test, p=0.1306. FIG. 4B, Representative images of spleen from CLT and CLT; AID−/− RDBC chimeras. Scale bars, 5 mm. FIG. 4C, Representative FACS analysis of splenic cells from control, CLT and CLT; AID−/− RDBC chimeras. Surface markers assayed are indicated on the right. FIG. 4D, Southern blotting analysis of EcoRI-digested genomic DNA of samples from spleen (S), liver (L) and purified splenic B cells (B) samples from the control, CLT and CLT; AID−/− RDBC chimeras. Other details of the analysis are the same as described for FIG. 2.

FIGS. 5A-5B Transplantation of LMP1-driven B cell population expansions derived from CLT and CLT; AID−/−RDBC chimeras. FIG. 5A, Representative images showing the spleens and livers of wild-type (WT,C57BL/6×BALB/c, F1) and RAG2−/−; γc−/− mice transplanted with the indicated donor clonal B cell expansions. Mice were analyzed in 3-9 weeks after transplantation. Scale bar, 5 mm. FIG. 5B, Southern blotting analysis on EcoRI-digested genomic DNA of samples separated from spleen (S) and liver (L) of the indicated mice. 1^{st} and 2^{nd} labels indicate the primary and transplanted tumors, respectively. Other details are as described in legend to FIG. 1.

FIG. 6 Flow cytometry measurements of peripheral blood B and T cells in indicated CLT RDBC chimeras and control mice. Peripheral blood cells from three week old mice were assayed expression the indicated B and T cell surface markers (bottom left) by flow cytometry.

FIG. 7 Comparative measurement of splenic and peripheral blood B-cell populations in the indicated CLT RDBC chimeras and control mice. Splenic and Peripheral blood cells from 6-8 week old mice were assayed for expression the indicated B and T cell surface markers (bottom left) by flow cytometry. 6-8 week old mice were examined by Flow Cytometry.

FIGS. 8A-8B CLT; AID−/− mice generated from germline breeding. FIG. 8A, Kaplan-Meier curves showing survival of mice within the CLT; AID+/− or CLT; AID+/+ and CLT; AID−/− cohorts. Cohorts of 8 CLT; AID+/−, 2 CLT; AID+/+ and 5 CLT; AID−/− germline mice are plotted together as controls. Ns means statistically not significant. FIG. 8B, Southern blotting analysis on EcoRI-digested genomic DNA of samples separated from spleen (S) and liver (L) of the indicated CLT; AID+/−, CLT; AID+/+, CLT; AID−/− and control mice using JH4-3 as the probe. Other details are as described in the legend to FIG. 2.

FIG. 9 Spleen size distributions of indicated CLT RDBC chimera cohorts. Bars indicate the mean value and SEM. N.s. means statistically not significant between two indicated groups as determined by a t test.

FIGS. 10A-10B Southern blotting analyses of B-cell expansions from CLT (FIG. 10A) and CLT as well as CLT; AID−/− RDBC (FIG. 10B) chimeras. DNA from the indicated control and chimera DNA samples were assayed for rearrangements of the 6.2 kb EcoR1 fragment containing the IgH JH region. Other details are as described in the legend to FIG. 2.

FIGS. 11A-11B Lsamp gene in ATM^{-/-} ROSA^{GR-l-Scel} c-Myc^{25XI-Scel} NSPCs harbors intragenic rearrangements. FIG. 11A, illustrating junction distribution around 4.2 Mb of the endogenous DSB cloning site (dash line). Arrowhead: translocation cloning primer. Lsamp gene region is shaded. 28 HTGTS junctions (top panel) were recovered from three independent HTGTS libraries. FIG. 11B, enlarged view of Lsamp gene (showing on top) and intragenic prey junctions (Box). E: exons of Lsamp. Junctions in centromere-to-telomere orientation (+) are peaks above the x-axis; junctions in telomere-to-centromere orientation are peaks below the x-axis. Bin size, 11 kb. Arrowhead: junctional cloning primer. Outcomes of translocation fall in different quadrants are shown. Intragenic Lsamp rearrangement lead to (1) deletions in Intron 2, indicated by black stars at (+) orientation and (2) potential alternative Lsamp variant templates including (a) deletion of Exon 2, (b) deletion of Exon 3, (c) deletion of Exon 3-4, and intragenic inversions involving an E3-E9 deletion, (d) and E4-E9 deletion (e), respectively.

FIGS. 12A-12B NestinCre⁺Xrcc4^{C/+}p53^{-/-} chimeric mouse develops medulloblastoma (MB). FIG. 12A, this panel illustrates the chimeric mouse generation scheme. NestinCre⁺Xrcc4^{C/+}p53^{-/-} ES cells were injected into wildtype blastocysts, which then were introduced into foster mothers to generate chimeric mice. FIG. 12B, MB in a NestinCre⁺Xrcc4^{C/+}p53^{-/-} chimeric mouse. The mouse brain shown came from a mouse euthanized at 11 weeks of age with MB symptoms including paralysis, weight loss, and eye closure. An asterisk marks the MB within the brain.

FIGS. 13A-13D, Optimization of Cas9/sgRNA expression in wildtype cerebellar granule cultures. FIG. 13A, immunofluorescent staining of cerebellar granule cells cultured in the presence of SHH for proliferation marker Ki57 and mature neuron marker NeuN at culture day 4.5. FIG. 13B, Immunofluorescent staining of Cas9-expressing cells transduced with Cas9/Chr15-Myc-sgRNA via lentivirus. FIG. 13C, Circos plot of the mouse genome divided into individual chromosomes howing the genome-wide HTGTS junction pattern of Chr15-Myc-sgRNA-mediated bait DSBs binned into 2.5-Mb regions (black bars); bar height indicates number of translocations per bin on a custom log scale. 3,753 junctions are plotted. Arrowhead: bait DSB/break site. FIG. 13D, junction distribution within 20 kb genomic region around the break site. 2,359 junctions are plotted in this figure. Dashed line: break site. Arrow: HTGTS junction cloning primers site. The c-Myc gene locus is highlighted. Prey sequences in centromere-to-telomere orientation (+) are peaks above the x-axis; prey sequences in telomere-to-centromere orientation (−) are peaks below the x-axis. The majority of junctions around the break site are enriched at the upper-right resection quadrant (75.75%) similar to what is found in other cell types.

### DETAILED DESCRIPTION

Described herein are rapid, reliable methods for generating mice (or other species) that develop tumors of known genotype with respect to two or more mutated, tumor-associated genes using blastocyst complementation.

As but one example, described herein are rapid methods for generating a transgenic mouse lymphoma model, by using a blastocyst complementation system, e.g., a RAG-2 blastocyst complementation system. Also described herein are rapid methods for generating a mouse model of medulloblastoma using a blastocyst complementation system. Also provided herein are transgenic mice made by such methods and use of the mice as a screening system for anti-cancer agents (including, but not limited to anti-lymphoma treatments or anti-medulloblastoma treatments).

### Definitions

The term “blastocyst complementation” as used herein refers to a technique for creating a chimeric animal in which injection of multipotent or pluripotent cells, such as ES cells and iPS cells, into an inner space of a blastocyst stage fertilized egg forms a chimeric animal when implanted into a female for gestation (e.g., pseudo-pregnant or pregnant female).

As used herein, the term “complex tumor mouse model” refers to a tumor mouse model having mutations in multiple independently segregating alleles. Such a compound mutant model can make it impractical to incorporate additional genetic modifications, since one or more desired mutations may reside on more than on chromosome, therefore requiring extensive backcrossing. In one embodiment, the complex tumor mouse model cannot be maintained as a pure strain, for example, due to frequent death or reproductive inefficiencies as a result of the tumor phenotype.

The term “RAG-2 deficient blastocyst complementation” refers to a type of blastocyst complementation in which an ES or iPS cell is injected into blastocysts from RAG-2 deficient mice to generate a chimeric mouse comprising ES- or iPS-cell derived lymphocytes.

The term “chimeric blastocyst” as used herein refers to a blastocyst that comprises cellular material from an ES or iPS cell(s) derived from a different source than that of the blastocyst. Such a chimeric blastocyst can be produced by, in addition to an injection method, utilizing a method such as a so-called “agglutination method” in which embryo+embryo, or embryo+cell are closely associated with each other in culture to produce a chimeric blastocyst. Further, the relationship between a recipient embryo and a cell to be transplanted can be an allogeneic relationship or a xenogeneic relationship.

As used herein the term “chimeric lymphocytes” refers to a lymphocyte having a genotype derived from a donor cell in an animal generated by blastocyst complementation (e.g., ES or iPS cell) and not from the host blastocyst. Such lymphocytes are considered to be “chimeric lymphocytes” in that they are not derived from the blastocyst itself and result in a chimeric phenotype in the animal with respect to the lymphocyte genotype.

As used herein, the term “modified genome” or “genetic mutation” refers to the introduction of an exogenous gene, an insertion, a deletion, a translocation, a site-specific mutation or an inversion into the genome of e.g., a pluripotent stem cell.

As used herein, the term “substitution, addition, and/or deletion” of a polypeptide or polynucleotide refers to substitution, addition, or removal of an amino acid or a substitute thereof, or a nucleotide or a substitute thereof, in an original polypeptide or polynucleotide, respectively. Techniques for generating these substitution, addition and/or deletion changes are known in the art, and examples of the techniques include a site-directed mutagenesis and the like. These changes in a reference nucleic acid or polypeptide can occur anywhere within the desired polynucleotide or polypeptide, for example, at the 5′-terminal or 3′-terminal of the nucleic acid, at the amino terminal or the carboxy terminal site of the amino acid sequence of the polypeptide, or at any site between those terminal sites so that the changes are present individually between residues of the reference sequence, as desired.

As used herein, the term “latent viral gene” refers to a viral gene that is expressed during a period of dormancy of the virus within a host cell and may function to prevent removal of the viral genome by the host's immune system or degradation of the viral genome by enzymes.

As used herein, the term “faster than conventional back-crossing methods” means that an equivalent mouse model is produced at least one month faster than the time necessary for the mouse model to be generated using conventional back-crossing methods. In some embodiments, the term “faster than conventional back-crossing methods” means at least 2 months faster, at least 6 months faster, at least 9 months faster, at least 1 year faster, at least 1.5 years faster, at least 2 years faster, at least 2.5 years faster, at least 3 years faster, or more as compared to conventional back-crossing methods.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment of the invention.

### Blastocyst Complementation

One approach to generate chimeric mice from embryonic stem (ES) cells is blastocyst microinjection, where one or more ES cells, for example, ES cells carrying a targeted genetic manipulation, are microinjected into a host blastocyst, which is then transferred to a foster mouse to generate ES cell-chimeric pups. In conventional blastocyst injections, a normal, diploid host blastocyst is injected with the desired ES cell population and both the injected ES cells and the inner cell mass (ICM) cells of the host blastocyst contribute to tissues of the chimeric mouse developing from such a blastocyst. Injected blastocysts can be transferred to foster mice to generate chimeric pups. If blastocyst injection is performed to produce a mouse line from the injected ES cells, the chimeric mice obtained from the blastocyst can be bred and, if the ES cell contributed to the germline of the chimeric mouse, ES cell-derived offspring can then be used to found an ES cell-derived mouse line, such as a line carrying a desired genetic modification. Host blastocysts used for the production of chimeric mice are typically derived from a mouse strain that is different from the strain of the injected ES cell line, which facilitates recognition of chimeric mice and offspring of chimeric mice by phenotype (e.g. coat color) or genotype of chimeric offspring. Same-strain injections are feasible as well, but require more effort in characterizing the generated mice.

For example, mice carrying a desired genetic modification can be propagated by breeding this “germline” chimera, thus generating pups in which all cells carry one ES cell-derived set of chromosomes. Genetic screening, for example, for a genetic modification introduced into the ES cells, and further breeding of the desired offspring of a germline chimera can then be performed to create a transgenic mouse strain. Such mice can be used, e.g., to study therapeutic approaches that target that tumor type, including but not limited to use to screen the efficacy of potential anti-tumor agents.

In one embodiment, the ES cell used in blastocyst complementation is obtained from a complex tumor mouse model. In another embodiment, an iPS cell derived from a complex tumor mouse model is used with methods for blastocyst complementation e.g., to generate a chimeric animal that recapitulates characteristics of the complex tumor mouse model.

Tetraploid blastocyst complementation is a special type of blastocyst injection approach, where diploid ES cells are injected into a tetraploid host blastocyst. Such tetraploid host blastocysts are typically generated by fusing the blastomeres of a diploid 2-cell stage embryo, thus generating an embryo made of one tetraploid cell. Tetraploid embryos are then cultured to the blastocyst stage and injected with diploid ES cells. The tetraploid cells of the host blastocyst form the extraembryonic tissues, such as the placenta, but show no significant contribution to tissues of the developing mouse. Accordingly, virtually all cells, including germ cells, of mice generated by tetraploid blastocyst complementation are derived from the injected diploid ES cells. Such mice can directly be used for phenotypic characterization, or as founder animals for a transgenic mouse strain.

### RAG-2 Deficient Blastocyst Complementation System

RAG-2 deficient mice are viable but fail to produce mature B or T lymphocytes even at several months of age due to an inability to initiate VDJ rearrangement leading to a severe combined immunodeficiency phenotype. This immunodeficiency phenotype is the only obvious abnormality detected in RAG-2 mutant mice, indicating the VDJ recombinase activity, per se, is not required for development of cells other than lymphocytes.

Researchers have used the RAG-2 deficient mouse to evaluate the function of genes in the differentiation and/or function of lymphocytes by generating chimeric mice. For example, injection of normal ES cells into RAG-2 deficient blastocysts leads to the generation of somatic chimeras with mature B and T cells derived from the injected ES cells (Chen et al. (1993) PNAS 90:4528-4532). This system provides a rapid and efficient means to elucidate gene function under normal developmental conditions. The system is ideal for analyzing the function of a gene in lymphocytes that may also be required in many different cells types and whose ablation can lead to early embryonic lethality.

While this system is typically used for determining normal lymphocytic development, it can also be used as described herein as a rapid and efficient means for generating a transgenic mouse lymphoma model.

Thus, the RAG-2 deficient mouse model and the transgenic mouse lymphoma model described herein can be used in screening anti-cancer agents. For example, potential drugs or known drugs can be administered to the tumor-bearing or transgenic animals and the efficacy of such drugs could be evaluated. Also, different treatment protocols can be tested in the transgenic animals.

In some embodiments, the RAG-2 deficient blastocyst complementation is performed using an ES or iPS cell that comprises a viral gene, for example, a viral latent gene. The viral gene can be derived from e.g., human immunodeficiency virus, cytomegalovirus, Herpes virus, Epstein Barr virus, another lymphotrophic virus, Hepatitis virus and the like which can infect lymphocytes. The transgenic mice generated using the methods described herein can be useful for identifying or evaluating candidate drugs against the viral infection.

### Pluripotent Stem Cells

Blastocyst complementation can be performed using any pluripotent stem cell, provided that the pluripotent stem cell does not otherwise interfere with normal development of the mouse, except changes in development initiated by the desired modified genome of the chimeric cells. Examples of a “pluripotent stem cell” that can be used in the methods described herein can include: an egg cell; an embryonic stem cell (ES cell); an induced pluripotent stem cell (iPS cell); a multipotent germ stem cell (mGS cell); and the like. In some embodiments, the genome of the pluripotent stem cell used in a blastocyst complementation method is modified, for example, comprises a gene that is expressed under the control of a tissue-specific promoter. In some embodiments, an exogenous gene, such as Cre recombinase, is present in the genome of the pluripotent stem cell. In other embodiments, genes can be flanked by lox sites to be knocked out under certain conditions, e.g., after a specific point in development, or in response to an inducing agent.

Embryonic Stem Cells:

In one embodiment, the pluripotent stem cell is an embryonic stem cell. Embryonic stem cells and methods of their retrieval are well known in the art and are described, for example, in Trounson A O (Reprod Fertil Dev (2001) 13: 523), Roach M L (Methods Mol Biol (2002) 185: 1), and Smith A G (Annu Rev Cell Dev Biol (2001) 17:435). The term “embryonic stem cell” is used to refer to the pluripotent stem cells of the inner cell mass of the embryonic blastocyst (see e.g., U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). In one embodiment, the embryonic stem cells are isolated from the embryo of a complex tumor mouse model.

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. The single blastomere cell is co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli.

In one embodiment, the ES cell is obtained from a tumor mouse model, such as the CLT mouse model. CLT mice have conditional B cell LMP1 expression and genetic elimination of α/β and γ/δ T cells, and die early in association with B cell lymphoproliferation and lymphomagenesis.

Induced Pluripotent Stem Cells:

iPSCs resemble ES cells as they restore the pluripotency-associated transcriptional circuitry and much of the epigenetic landscape. In addition, iPSCs satisfy all the standard assays for pluripotency: specifically, in vitro differentiation into cell types of the three germ layers, teratoma formation, contribution to chimeras, germline transmission (Maherali and Hochedlinger, 2008), and tetraploid complementation (Woltjen et al., 2009). An induced pluripotent stem cell for use with the methods described herein can be generated using any method known in the art to reprogram somatic cells from an animal with a complex tumor genotype to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent. The specific reprogramming approach or method used to generate pluripotent stem cells from somatic cells is not critical to the methods described herein. Thus, any method that reprograms a somatic cell to the pluripotent phenotype would be appropriate for use in the methods described herein.

Reprogramming methodologies using defined combinations of transcription factors have been described for generating induced pluripotent stem cells. Yamanaka and Takahashi converted mouse somatic cells to ES cell-like cells with expanded developmental potential by the direct transduction of genes encoding Oct4, Sox2, Klf4, and c-Myc (Takahashi and Yamanaka, 2006). Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5; 7(5):618-30). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

### Generation of Complex Mouse Tumor Models

In one embodiment, provided herein are methods for generating an ES cell from a complex mouse tumor model and the subsequent use of the ES cell for blastocyst complementation to generate cohorts of mice that will develop the same tumor (e.g., a tumor that, at a minimum, has the same set of cancer-related genetic defects or changes as the animal from which the pluripotent cell (e.g., ES cell) was obtained). This process obviates the need for expensive and complicated mouse breeding to generate or maintain particular cancer models. The methods described herein also permit the efficient and relatively inexpensive generation of additional mutations into the complex tumor genetic background that could modify the behavior of the tumor or the ability of the host to interact (e.g., eradicate it).

Recapitulation of a complex mouse tumor model can be performed by providing an ES cell or iPS cell derived from the complex mouse tumor model and subsequent blastocyst complementation. Alternatively, introduction of additional genetic mutations into complex mouse genetic backgrounds can easily be performed by providing an ES cell or an iPS cell comprising at least one genetic mutation and its use in subsequent blastocyst complementation.

CRISPR/Cas:

In one embodiment, a genetic mutation is introduced to a genome of a pluripotent stem cell (e.g., a pluripotent stem cell derived from a complex murine tumor model) using CRISPR/Cas targeted genome editing. The CRISPR (clustered regularly interspaced short palindromic repeats)/Cas9 (CRISPR-associated protein-9) system has emerged as an efficient tool to mutate, delete and insert genomic DNA sequences in a site-specific manner. In CRISPR-mediated genome editing, Cas9 protein is directed to cleave DNA by an associated single guide RNA (sgRNA) hairpin structure that can be designed to target almost any genomic site of interest. Site specific mutagenesis and targeted transgenesis are key applications for studying development and disease, and as such, the ability to easily edit any genomic locus is revolutionizing stem cell research. Methods for genomic editing using CRISPR/Cas are known in the art and are described, for example, in U.S. Pat. No. 8,871,445, which is incorporated herein by reference in its entirety.

Cre/Lox:

In one embodiment, Cre-Lox recombination is used to introduce a genetic mutation into the genome of a complex tumor model-derived pluripotent stem cell. Cre-Lox recombination is a site-specific recombinase technology that can be used to introduce deletions, insertions, translocations and inversions at specific sites in the genomic material of a cell. Briefly, the system consists of a single enzyme, Cre recombinase, that recombines a pair of short target sequences called the Lox sequences. Placing Lox sequences appropriately allows genes to be activated, repressed, or exchanged for other genes. The activity of the Cre enzyme can be controlled so that its expression is localized to a particular cell type or induced by an external stimulus like a chemical signal or a heat shock. Methods for modifying a genome of a cell using Cre/Lox recombination is well known to those of skill in the art and is not described in detail herein.

### In Vivo Screening Assays

Any of the tumor mouse models derived using the methods described herein can be utilized in an in vivo screening assay, for example to test and identify agents useful in the treatment of cancer. Screening assays as contemplated herein can be used to identify modulators, i.e., candidate or test compounds or agents (e.g., peptides, antibodies, peptidomimetics, small molecules (organic or inorganic) or other drugs) which have anti-cancer, anti-neoplastic, or anti-tumor activity.

The term “candidate agent” is used herein to mean any agent that is being examined for anti-cancer activity. Although the tumor-bearing transgenic animals generally used in a screening assay will identify previously unknown molecules that can act as a therapeutic agent, the screening described herein can also be used to confirm that an agent known to have such activity, in fact has the activity, for example, in standardizing the activity of the therapeutic agent. A candidate agent can be any type of molecule, including, for example, a peptide, a peptidomimetic, a polynucleotide, or a small organic molecule, that one wishes to examine for the ability to modulate a desired activity, such as, for example, anti-cancer activity.

Accordingly, the term “agent” as used herein in the context of screening means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In some embodiments, the nucleic acid is DNA or RNA, and nucleic acid analogues, for example can be PNA, pcPNA and LNA. A nucleic acid may be single or double stranded, and can be selected from a group comprising; nucleic acid encoding a protein of interest, oligonucleotides, PNA, etc. Such nucleic acid sequences include, for example, but not limited to, nucleic acid sequence encoding proteins that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide agent or fragment thereof can be, for example, but not limited to; mutated proteins; therapeutic proteins; truncated proteins, wherein the protein is normally absent or expressed at lower levels in the cell. Proteins of interest can be selected from a group comprising; mutated proteins, genetically engineered proteins, peptides, synthetic peptides, recombinant proteins, chimeric proteins, antibodies, humanized proteins, humanized antibodies, chimeric antibodies, modified proteins and fragments thereof.

In certain embodiments, the candidate agent is a small molecule having a chemical moiety. Such chemical moieties can include, for example, unsubstituted or substituted alkyl, aromatic, or heterocyclyl moieties and typically include at least an amine, carbonyl, hydroxyl or carboxyl group, frequently at least two of the functional chemical groups, including macrolides, leptomycins and related natural products or analogues thereof. Candidate agents can be known to have a desired activity and/or property, or can be selected from a library of diverse compounds. Also included as candidate agents are pharmacologically active drugs, genetically active molecules, etc. Such candidate agents of interest include, for example, chemotherapeutic agents, hormones or hormone antagonists, growth factors or recombinant growth factors and fragments and variants thereof. Exemplary of pharmaceutical agents suitable for use with the screening methods described herein are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all of which are incorporated herein by reference in their entireties. Also included are toxins, and biological and chemical warfare agents, for example see Somani, S. M. (Ed.), “Chemical Warfare Agents,” Academic Press, New York, 1992), the contents of which is herein incorporated in its entirety by reference. Candidate agents, such as chemical compounds, can be obtained from a wide variety of sources including libraries of synthetic or natural compounds, such as small molecule compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Synthetic chemistry transformations and protecting group methodologies (protection and deprotection) useful in synthesizing the candidate compounds for use in the screening methods described herein are known in the art and include, for example, those such as described in R. Larock (1989) Comprehensive Organic Transformations, VCH Publishers; T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof, the contents of each of which are herein incorporated in their entireties by reference.

With regard to intervention, any treatments which comprise anti-cancer or chemotherapeutic activity should be considered as candidates for human therapeutic intervention.

The present invention may be as defined in any one of the following numbered paragraphs:

1. A method for generating a transgenic mouse lymphoma model, the method comprising: (a) introducing an embryonic stem (ES) cell or induced pluripotent stem (iPS) cell comprising a modified genome to a RAG-2-deficient blastocyst to produce a chimeric blastocyst, and (b) implanting the chimeric blastocyst into a female mouse for gestation, thereby generating a transgenic mouse lymphoma model.

2. The method of paragraph 1, wherein the ES cell or iPS cell is derived from a tumor model mouse.

3. The method of paragraph 2, wherein the tumor model mouse is homozygous recessive for one or more tumor-related genes.

4. The method of paragraph 1, 2 or 3, wherein the modified genome comprises at least one insertion, deletion, or mutation.

5. The method of any one of paragraphs 1-4, wherein the genome modification comprises addition of an activated oncogene or a latent viral gene.

6. The method of any one of paragraphs 1-5, wherein the transgenic mouse comprises lymphocytes having the genotype of the ES or iPS cell.

7. The method of paragraph 6, wherein at least 50% of the lymphocytes in the transgenic mouse have a genotype derived from the ES or iPS cell.

8. The method of paragraph 5, wherein the latent viral gene is derived from the Epstein-Barr virus.

9. The method of paragraph 5, wherein the latent viral gene is LMP-1.

10. The method of any one of paragraphs 1-9, wherein the transgenic mouse comprises B and/or T cells derived from the ES or iPS cell.

11. The method of any one of paragraphs 1-9, wherein the method provides transgenic mice faster than conventional back-crossing methods.

12. A transgenic mouse lymphoma model made by the method of any one of paragraphs 1-11.

13. A RAG-2 deficient transgenic mouse lymphoma model, comprising: chimeric lymphocytes having a modified genome, wherein the genotype of the lymphoma is that of the chimeric lymphocytes.

14. The transgenic mouse of paragraph 13, wherein at least 50% of the lymphocytes in the transgenic mouse are chimeric lymphocytes.

15. The transgenic mouse of paragraph 13 or 14, wherein the modified genome comprises at least one insertion, deletion, or mutation.

16. The transgenic mouse of any one of paragraphs 13-15, wherein the modified genome comprises a latent viral gene or an oncogene.

17. The transgenic mouse of any one of paragraph 13-16, wherein the latent viral gene is derived from the Epstein-Barr virus.

18. The transgenic mouse of paragraph 17, wherein the latent viral gene is LMP-1.

19. The transgenic mouse of any one of paragraphs 13-18, further comprising at least one symptom of a B cell expansion or a B cell lymphoma.

20. The transgenic mouse of paragraph 19, wherein the at least one symptom is selected from the group consisting of: splenomegaly, splenic tumor nodules, hepatomegaly, and hepatic tumor nodules.

21. The transgenic mouse of paragraph 19, wherein the B cells comprise at least one cell surface marker profile selected from the group consisting of: CD19⁺, CD95/Fas⁺, B220^{low}, IgM⁺, Igk⁺, CD4⁻ and CD8⁻.

22. The transgenic mouse of any one of paragraphs 13-21, wherein the chimeric lymphocytes are derived from a tumor mouse model.

23. The transgenic mouse of any one of paragraphs 13-22, wherein the tumor mouse model is homozygous recessive for one or more tumor-related genes.

24. The transgenic mouse of paragraph 23, wherein the tumor mouse model comprises a genotype that is TCRβ^{-/-}δ^{-/-}.

25. The transgenic mouse of paragraph 22, wherein the tumor mouse model is the CLT mouse model.

26. A screening assay for identifying an anti-cancer agent for the treatment of lymphoma, the assay comprising: (a) determining the extent of B cell expansion or lymphoma in a RAG-2 deficient transgenic mouse, wherein the RAG-2 deficient transgenic mouse comprises chimeric lymphocytes having a modified genome and further comprises a lymphoma having a genotype of the chimeric lymphocytes, (b) administering a candidate anti-cancer agent to the RAG-2 deficient mouse, and (c) comparing the extent of B cell expansion or lymphoma in the treated RAG-2 deficient mouse, wherein a decrease in the extent of B cell expansion or lymphoma identifies the candidate as an anti-cancer agent for treatment of lymphoma.

27. The assay of paragraph 26, wherein the extent of B cell expansion or lymphoma is determined by measuring spleen size.

28. The assay of paragraph 26 or 27, wherein spleen size is determined using ultrasound technology.

29. A method for generating a complex mouse tumor model, the method comprising: (a) introducing an embryonic stem (ES) cell or induced pluripotent stem (iPS) cell derived from a complex mouse tumor model and having a modified genome to a donor blastocyst to produce a chimeric blastocyst, and (b) implanting the chimeric blastocyst into a female mouse for gestation, thereby generating a complex mouse tumor model.

30. The method of paragraph 29, wherein the tumor model mouse is homozygous recessive for one or more tumor-related genes.

31. The method of paragraph 29 or 30, wherein the modified genome comprises at least one insertion, deletion, or mutation.

32. The method of paragraph 31, wherein the genome modification comprises addition of an activated oncogene.

33. The method of any one of paragraphs 29-32, wherein the method provides transgenic mice faster than conventional back-crossing methods.

34. A complex mouse tumor model made by the method of any one of paragraphs 29-33.

35. A method for generating a medulloblastoma model mouse, the method comprising: (a) introducing a p53^{-/-}, Nestin-Cre/XRCC4-conditional murine embryonic stem cell or induced pluripotent stem cell to a murine blastocyst to produce a chimeric blastocyst; and (b) implanting the chimeric blastocyst into a female mouse for gestation, whereby resulting chimeric p53^{-/-}Nestin-Cre/XRCC4-conditional mice are predisposed to the development of medulloblastoma.

36. The method of paragraph 35, wherein resulting chimeric mice are XRCC4⁻ in neuronal tissue.

37. The method of paragraph 35 or 36, wherein the murine blastocyst is from a wildtype animal.

38. A method for generating a medulloblastoma mouse model, the method comprising:

(a) introducing a murine embryonic stem cell or induced pluripotent stem cell to a murine blastocyst to produce a chimeric blastocyst, wherein the embryonic stem cell or induced pluripotent stem cell is p53^{-/-} and carries a Cre-recombinase coding sequence operably linked to a neuronal-tissue-specific promoter and XRCC4 coding sequence flanked by loxP sites; and

(b) implanting the chimeric blastocyst into a female mouse for gestation, whereby resulting chimeric p53^{-/-}/Cre/XRCC4-conditional mice are predisposed to the development of medulloblastoma.

39. The method of paragraph 38, wherein resulting chimeric mice are XRCC4⁻ in neuronal tissue.

40. The method of paragraph 38 or 39, wherein the murine blastocyst is from a wildtype animal.

41. A medulloblastoma mouse model made by the method of any one of paragraphs 35-40.

### EXAMPLES

The Epstein-Barr virus (EBV) latent membrane protein 1 (LMP1) contributes to oncogenic human B cell transformation. Mouse B cells conditionally expressing LMP1 are not predisposed to B cell malignancies, as LMP1-expressing B cells are eliminated by T cells. However, mice with conditional B cell LMP1 expression and genetic elimination of α/β and γ/δ T cells (“CLT” mice) die early in association with B cell lymphoproliferation and lymphomagenesis. Generation of CLT mice involves in-breeding multiple independently segregating alleles. Thus, while introduction of additional activating or knock-out mutations into the CLT model is desirable for further B cell expansion and immunosurveillance studies, doing such experiments by germline breeding is time-consuming, expensive and sometimes unfeasible. To generate a more tractable model, the inventors generated clonal CLT ES cells from CLT embryos and injected them into RAG2-deficient blastocysts to generate chimeric mice, which, like germline CLT mice, harbor splenic CLT B cells and lack T cells. CLT chimeric mice generated by this RAG2-deficient blastocyst complementation (“RDBC”) approach die rapidly in association with B-cell lymphoproliferation and lymphoma. As CLT lymphomas routinely express the Activation-Induced Cytidine Deaminase (AID) antibody diversifier, the inventors tested potential AID roles by eliminating the AID gene in CLT ES cells and testing them via RDBC. It was found that CLT and AID-deficient CLT ES chimeras had indistinguishable phenotypes, showing that AID is not essential for LMP1-induced lymphomagenesis. Beyond expanding accessibility and utility of CLT mice as a cancer immunotherapy model, these studies provide a new approach for facilitating generation of genetically complex mouse cancer models.

### Example 1: Blastocyst Complementation Method for Generating Complex Tumor Models in Mice

### Materials and Methods

### Generation of CLT RDBC Chimeric Mice

The CLT ES cell line was derived from embryonic day 3.5 (E3.5) blastocysts resulting from a cross between CD19cre/+; TCRβ−/−δ−/− and LMP1stopFL/+; TCRβ−/−δ−/− mice according to an established protocol (19). The transgenic mice LMP1stopFL used were generated on a BALB/c background whereas other mouse strains including CD19-cre, TCRβ−/−, and TCRδ−/− were all on a C57BL/6 background (8). RAG2-deficient blastocyst injection and implantation were performed as described (17). For each RDBC injection, 6 to 8 ES cells were injected per RAG2-deficient blastocyst, and 26 to 28 injected blastocysts were implanted. Chimeric mice were assessed for extent of chimerism at 3 weeks of age by tail-PCR. All of the animal experiments were performed under protocols approved by the Institutional Animal Care and Use Committee of Boston Children's Hospital.

### ES Cell Targeting

Cas9/gRNAs targeting the AID locus were cloned into PX330 (Addgene plasmid 42230) as described (20). Multiple gRNA targeting sequences (PAMs) were used as follows: AID-A: GTAGGTCTCATGCCGTCCCT (TGG), AID-B: GCCGAAGTCCAGTGAGCAGG (AGG), AID-C: GGATTTTGAAAGCAACCTCC (TGG), and AID-D: GCGAGATGCATTTCGTATGT (TGG). The maintenance, transfection and screening of CLT ES cells for targeting experiments were performed as described (21).

### Flow-Cytometry Analysis

Single-cell suspensions from the spleen and liver of CLT and CLT; AID−/− RDBC chimeras and control mice were stained with each of the following sets of anti-mousemonoclonal antibodies: anti-B220-PE-Cy5 (eBioscience™ Inc.) and anti-IgM-FITC (eBioscience™ Inc.); anti-B220-APC (eBioscience™ Inc.) and anti-IgK-PE (BD Biosciences™); anti-CD19-FITC (BD Biosciences™) and anti-CD95/Fas-PE-Cy7 (BD Biosciences™); or anti-CD8a-APC (BD Biosciences™) and anti-CD4-FITC (eBioscience™ Inc.). FACS data was acquired using the FACSCalibur Flow Cytometer equipped with CellQuest software (BD Biosciences™) and analyzed with FlowJo software (TreeStar™).

### Southern Blotting

Southern blotting was performed with 5-10 μg of genomic DNA isolated from the spleen and liver of CLT and CLT; AID−/− RDBC chimeras and control mice or CLT ES cell clones as described previously (22). The JH4-3 probe is a 1.6 kb HindIII/EcoRI fragment downstream of JH4. The AID probe is a 585 bp EcoRI fragment located at 5′ arm of AID locus. A PCR fragment comprising exosc3 exon 3 was used as a loading control probe.

Real-Time qRT-PCR

Total RNA was isolated with TriPure Isolation Reagent (Roche™) and reverse-transcribed by M-MLV Reverse Transcriptase (Invitrogen™) with oligo (dT). qRT-PCR was performed using Power SYBR Green PCR Master Mix (Applied Biosystems™) on 7300 Real Time PCR System (Applied Biosystems™) with AICDA specific primers: GGGCCAAGGGACGGCATGAG and CCCGGGTCCAGGTCCCAGTC. The HPRT gene was detected in parallel and used as the internal control with primers GTCATGCCGACCCGCAGTC and GTCCTGTCCATAATCAGTCCATGAGGAATAAAC.

### Western Blotting

Cell suspensions were lysed on ice for 5 min with lysis buffer (50 mM Tris-HCl, pH7.4, 150 mM NaCl, 1% NP40, 5 mM EDTA, and 1 mM PMSF) supplemented with 1×Protease Inhibitor Cocktail (Roche™). After lysis of nuclei with final concentration of 500 mM NaCl and elution of genomic DNA by adding equal volume of water and centrifuging at 13,000 rpm for 10 min, the supernatant was boiled with 1×SDS loading buffer, separated by SDS-PAGE, and probed with an AID polyclonal antibody that has been described (23). Tubulin was detected in parallel with anti-α-tubulin antibody (Sigma, T5168) and used as a loading control.

### Tumor Transfer

In each set of the tumor transfer experiments, at least 5×10⁵ cells from 4 independent B cell expansions derived from either CLT or CLT; AID−/− RDBC chimeras were transferred via intravenous (i.v.) injection into immunodeficient (RAG2−/−; γc−/−) and immunocompetent (C57BL/6×BALB/c, F1) mice. Recipients were observed for evidence of tumor formation and further characterization performed as outlined above for analyses of primary chimeras.

### Results

CLT mice provide a very attractive model for studies of EBV-related pathologies and immune surveillance (8,9). However, this compound mutant model cannot be maintained as a pure strain due to frequent death of CLT mice from B cell expansion/lymphoma before breeding age. Moreover, the complexity of the model, which is based on multiple independently segregating alleles, makes it impractical with respect to incorporating additional genetic modifications. Indeed some desired mutations may reside on one of the 4 mutant chromosomes, requiring extensive back-crossing for their introduction into the model. The inventors aimed to develop a CLT mouse model into which new mutant alleles could be easily introduced.

For this purpose, the inventors used the RAG2-deficient blastocyst complementation (RDBC) approach (17). Development of B and T cells is abrogated at the progenitor stage in RAG2-deficient mice, as RAG2 is essential for the V(D)J recombination process that assembles antigen receptor variable region exons (18). Thus, when V(D)J competent ES cells are injected into RAG2-deficient blastocysts, all B and T cells derive from the injected ES cells, allowing use of ES cells containing other types of mutations to test effects on lymphocyte development and function. To generate the ES cell based CLT model, the inventors established a CLT embryonic stem (ES) cell line which was then used for RDBC, resulting in chimeric mice that died early with phenotypes indistinguishable from those of germline CLT mice. To further test the utility of the model, the inventors also tested CLT cells in which both copies of the AID gene were eliminated via a Cas9/gRNA nuclease strategy. The AID gene lies on chromosome 6 along with the LMP1 knock-in (Rosa26 gene) and the TCRβ locus.

Generation of CLT ES Cells and their Use for RDBC

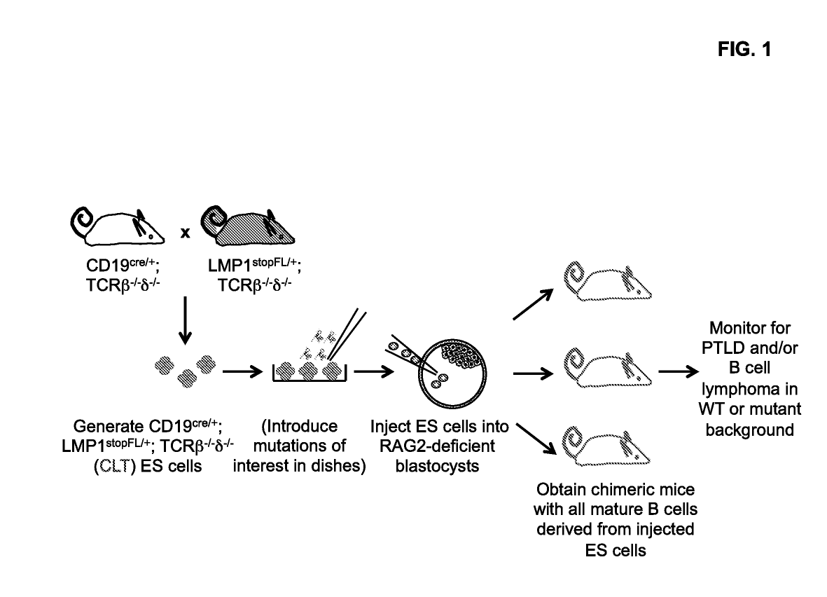

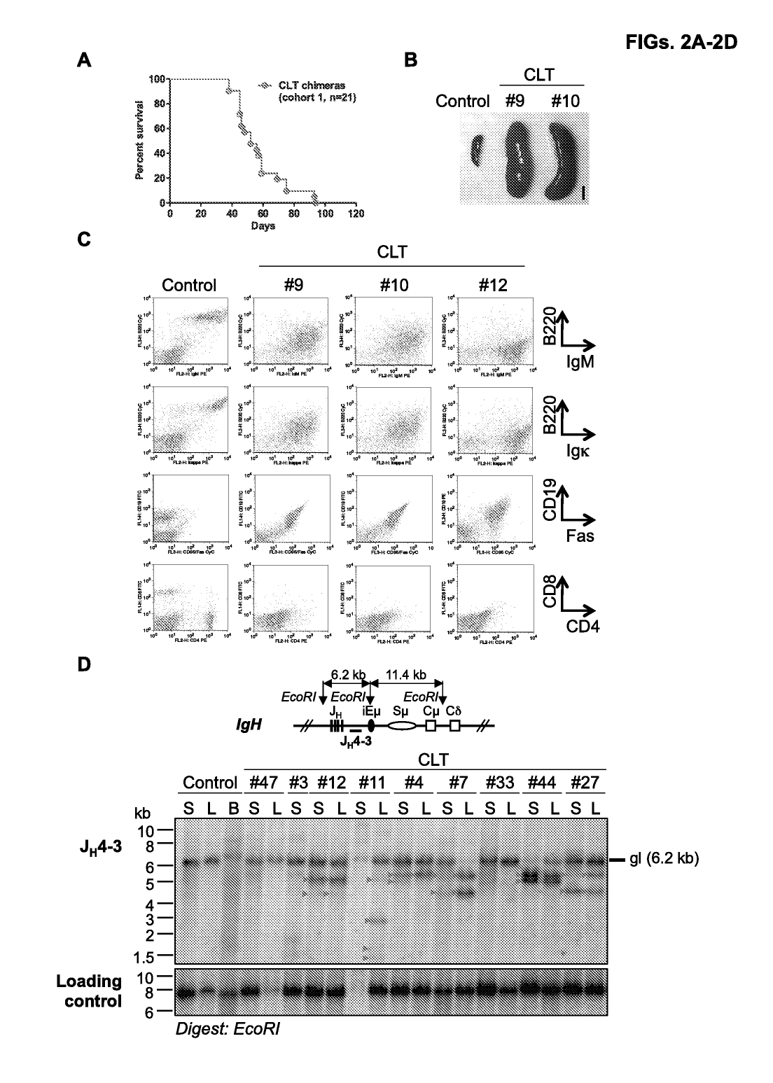

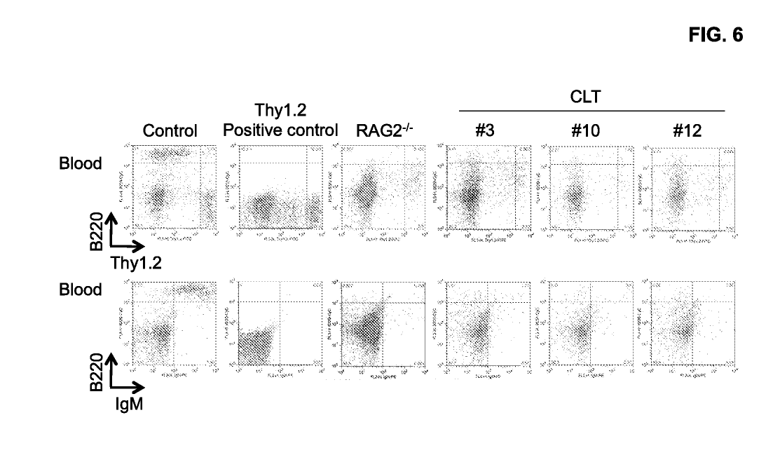

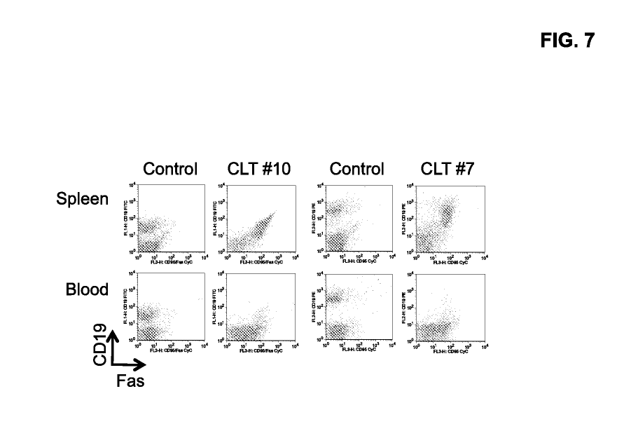

Given the difficulty in introducing additional genetic alterations into the CLT background by standard breeding approaches, the inventors sought to develop a more efficient strategy to generate such models through the use of the inventors' previously developed RDBC approach. For this purpose, the inventors generated an ES cell line from CLT embryos derived from crosses between CD19cre/+; TCRβ−/−δ−/− and LMP1stopFL/+; TCRβ−/−δ−/− mice. The inventors then injected the CLT ES cells into RAG2−/− blastocysts to generate chimeric mice in which all peripheral lymphocytes must derive from the injected ES cells (FIG. 1). The standard assay for the extent of chimerism in the RDBC chimeras made with ES cells expected to support normal B and/or T cells development is a standard flow cytometry measurement of peripheral blood B and T cell numbers (17). However, it was found that three week old CLT RDBC chimeras had not only no peripheral T cells, as expected due to the TCRβ−/−δ−/− mutations, but also very few circulating B cells (FIG. 6). Yet, up to 10 chimeras from each injection had a strong contribution from the CLT ES cells (up as much as 75% contribution) as assessed by genotyping of the CD19-cre transgene integrated in the CLT ES cells by tail-PCR of three weeks old pups. Thus, this finding, coupled with the finding of expanded LMP1+B cell populations in CLT RDBC chimera spleens (see below), indicates a potential defect in the recruitment of LMP1+ B cells into the peripheral blood, a possibility that had not been previously examined in germline CLT mice (8). Indeed, examination of 6-8 week old CLT RDBC chimeras confirmed robust splenic CLT B cells numbers but few B cells in the peripheral blood (FIGS. 2C and 7).

RDBC Chimeras Generated with CLT ES Cells Develop Typical LMP1-Driven Polyclonal B Cell Expansions and B-Cell Lymphomas

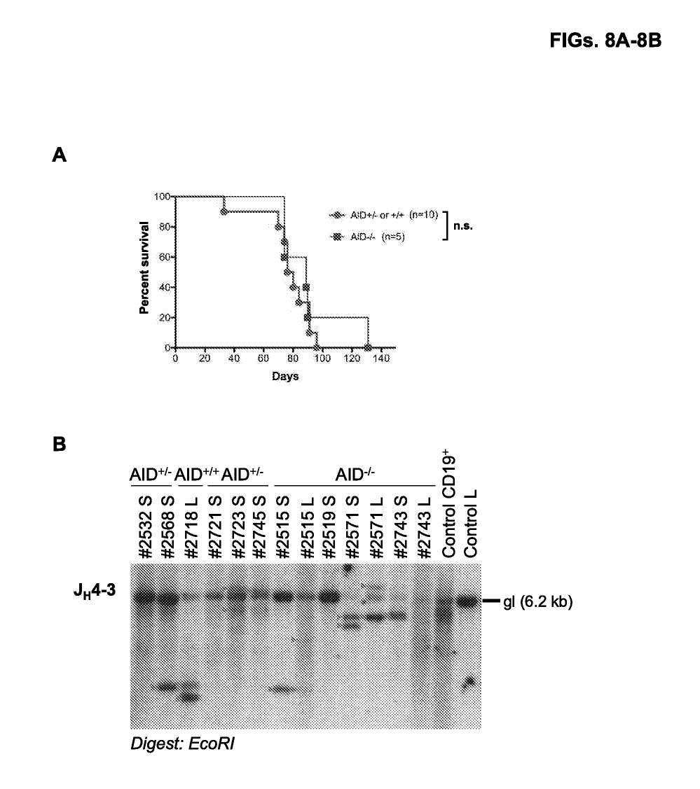

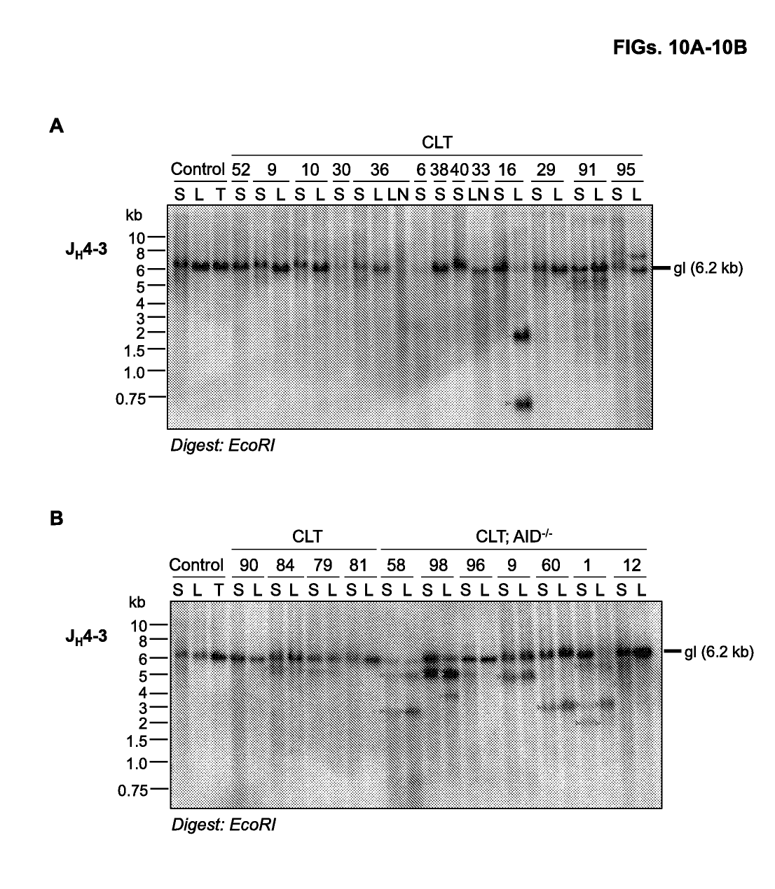

Of a cohort of 21 CLT RDBC chimeras maintained for analysis, all died or were sacrificed with extremely large spleens as determined by ultrasound by 13 weeks of age, with 50% of the animals dead or sacrificed by 7 weeks (FIG. 2A; Table 1). A second cohort of 8 CLT RDBC control chimeras had a very similar survival curve (see below). Indeed, the survival curves of the two cohorts of CLT RDBC chimeras analyzed were remarkably similar to those of germline CLT mice (8) (FIG. 8A), with perhaps a slightly less sharp curve perhaps due to some variation in the extent of chimerism versus constant full contribution of mutant cells in germline mice. The vast majority of the CLT RDBC chimeras (19 of 21; the other two mice were died before they could be analyzed) presented with splenomegaly sometimes accompanied by apparent tumor nodules, with spleen sizes reaching 27 mm on average (FIGS. 2B and 10). The dead or sacrificed CLT RDBC chimeras also, in several cases, showed hepatomegaly accompanied by tumor nodules (data not shown). FACS analysis demonstrated that the massive splenic cell expansions were of B cell origin as the vast majority of cells within them were CD19+, CD95/Fas+, B220low, IgM+, and Igk+, but CD4− and CD8− (FIG. 2C). Moreover, expression of CD95/Fas in these B cell expansions confirmed prior findings that ectopic LMP1 expression is accompanied by the up-regulation of CD95/Fas, which indeed has been used as a surrogate marker for LMP1 expression (4,8,24). Thus, the surface marker expression pattern of the splenic B cell expansions in CLT RDBC chimeras is the same as that found in the germline CLT model (8).

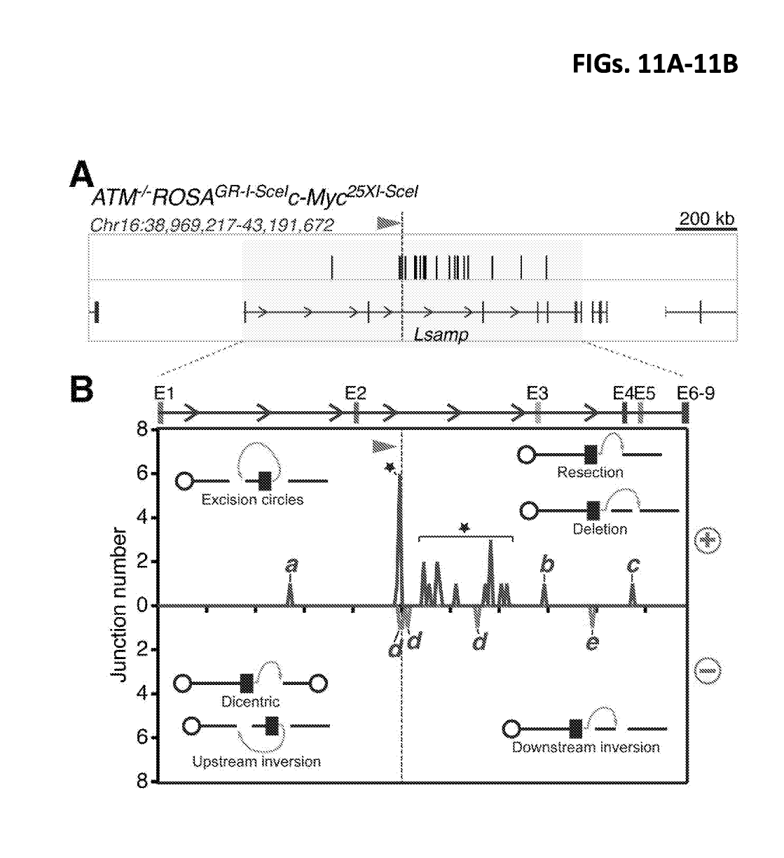

To further characterize the nature of the B cell expansions in CLT RDBC chimeras, the inventors assayed DNA from enlarged spleens and livers of these chimeras for rearrangements of the immunoglobulin heavy chain locus (IgH) JH region by asouthern blotting strategy that employed EcoR1-digested DNA and a JH4-specific probe (FIG. 2D and FIG. 11). Consistent with findings from the germline CLT mice (8), samples from chimeras terminally ill with enlarged spleens and/or livers before approximately 7 weeks of age generally had either no obvious IgH rearranged bands or weak polyclonal rearrangements, as exemplified by chimeras #47 (38 days), #52 (38 days), #9 (45 days), #10 (45 days) and #3 (46 days) (FIGS. 2D and 11A). However, chimeras that were terminally ill with enlarged spleens or livers at later ages often showed clonal IgH rearrangements, as exemplified by chimeras #4 (57 days), #7 (59 days), #44 (59 days), #16 (75 days) and 27 (94 days) (FIGS. 2D and 11A). Moreover, as observed in the germline CLT model, several CLT RDBC chimeras had clonal IgH rearrangements that overlapped in size in spleen and liver samples (e.g. chimera samples #12, #7, #44, #27 and #91 (FIGS. 2D and 11A), indicating that they represent expansions of the same clonally derived B cells, mostlikely of splenic B cell origin.

### AID Ablation has No Obvious Effects on LMP1-Driven Lymphomagenesis and B-Cell Lymphomas

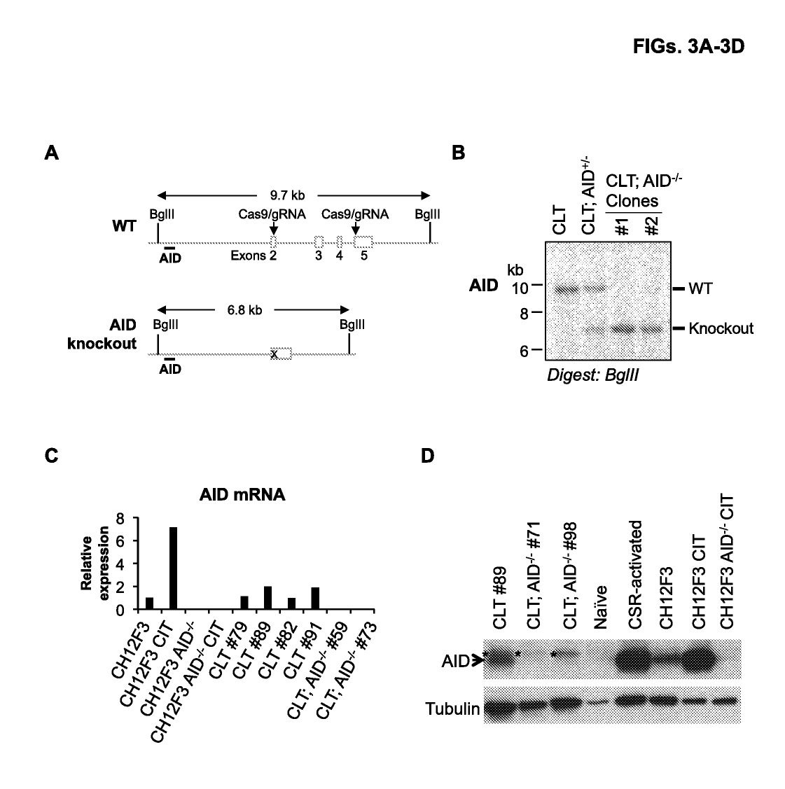

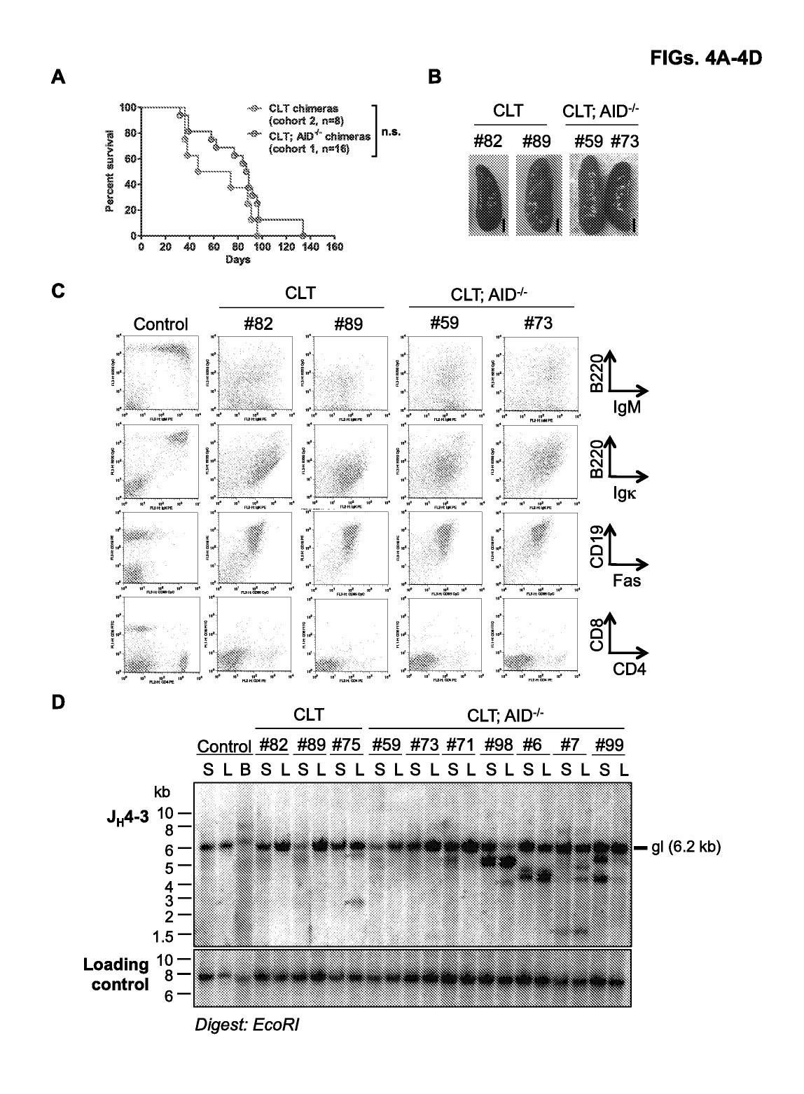

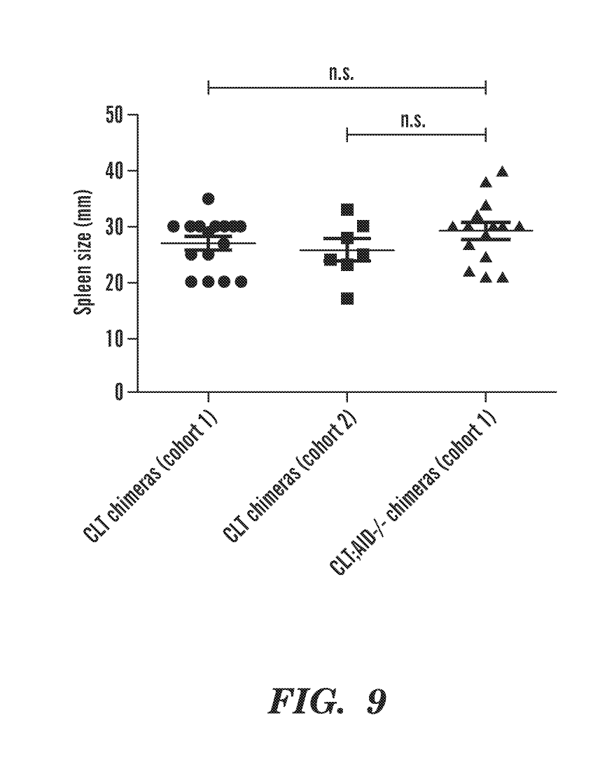

To explore potential roles of AID expression in CLT lymphomagenesis, the inventors generated CLT mice in an AID-deficient background by multiple rounds of breeding. However, because the AICDA gene, LMP1 knock-in allele (Rosa26 gene) and the TCRβ locus all lie on chromosome 6 of mouse genome, compound mutant mice were obtained at only very low frequency through sister chromatid crossovers during meiosis in the germline model. By this approach, at least two years of breeding was required to generate just 5 CLT; AID−/− mice, 8 CLT; AID+/− and 2 CLT; AID+/+ control mice. Although both groups of mice developed CLT B cell expansions and, in some cases, clonal B cell lymphomas, the CLT; AID−/− cohort was too small for in depth comparison of onset times and other aspects of the expansions/tumors (FIG. 9); whereas the control cohort of CLT; AID+/− and CLT; AID+/+ mice appeared similar in all respects to CLT model reported previously (8) (FIG. 9). To more rapidly generate larger cohorts of AID−/− CLT mice for analysis, the inventors employed CRISPR/Cas9-mediated genome editing (20) to delete both AID alleles in CLT ES cells (FIGS. 3A, 3B); and then used the AID−/− CLT ES cells along with CLT controls for RDBC. By this approach the inventors generated a cohort of 16 CLT; AID−/− RDBC chimeras (based on two RDBC injections of two independent AID−/− CLT ES cell clones). At the same time a cohort of 8 control CLT RDBC chimeras were generated for comparison. The WT and AID−/− CLT RDBC cohorts showed similar survival curves (FIG. 4A). Likewise, 7 of 8 CLT and 15 of 16 CLT; AID−/− RDBC chimeras presented with splenomegaly, as determined by ultrasound, between 5-13 weeks and 5-19 weeks of age, and with average spleen sizes of 26 mm and 29 mm, respectively (FIGS. 4B and 10). Hepatomegaly and tumor nodules within the liver were also occasionally observed in both CLT and CLT; AID−/− RDBC chimeras (data not shown). Analysis of expanded CLT splenic B-cell populations from these CLT; AID−/− RDBC chimeras confirmed lack of AID expression at both RNA and protein levels (FIGS. 3C, 3D). Further analyses also showed that CLT; AID−/− expansions and tumors had the same surface marker expression (e.g. FIG. 4C) and overall patterns of polyclonal or clonal JH rearrangements as those of CLT RDBC chimeras (FIGS. 4D and 11). Taken together, these analyses demonstrate that CLT; AID−/− RDBC chimeras develop B cell expansions and tumors over the same general time period with the same general characteristics as CLT RDBC chimeras and CLT germline mice.

### Propagation of CLT and CLT; AID−/− B Cell Expansions in Immunodeficient Recipients

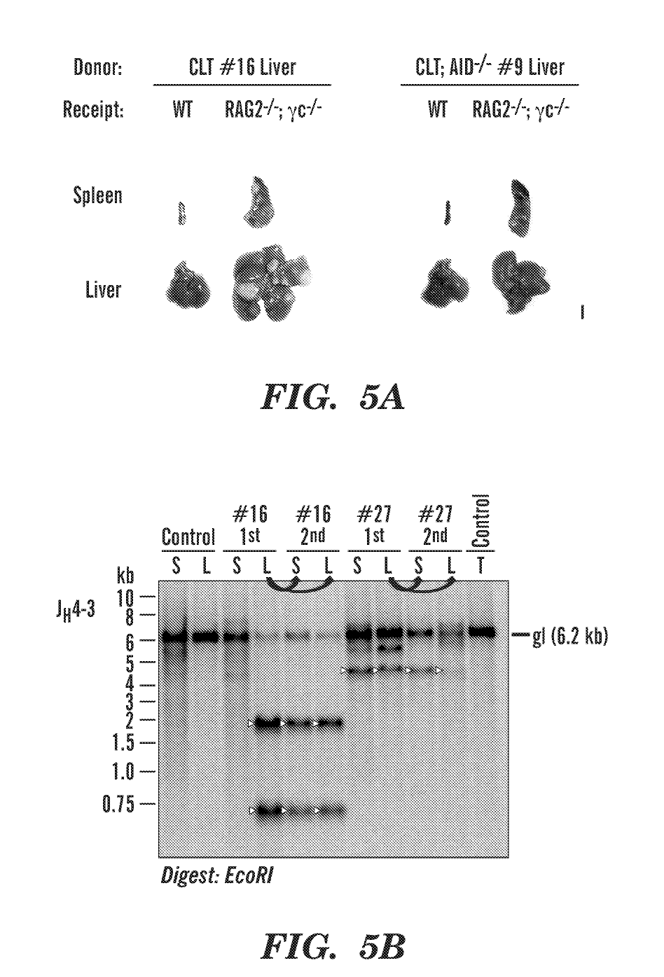

To confirm that the B cell expansions in both CLT and CLT; AID−/− RDBC chimeras contain transformed cells, the inventors tested the transferability of expansions from 4 CLT and 4 CLT; AID−/− RDBC chimeras into immunodeficient RAG2−/−; common γ chain−/− (RAG2−/−; γc−/−) mice that lack B, T, and NK cells) or into immunocompetent (C57BL/6×BALB/c, F1) recipients. Many immunodeficient recipients injected with B cell expansions from CLT or CLT; AID−/− RDBC chimeras, respectively, developed enlarged spleens and in some cases, livers, with distinct tumor nodules (FIG. 5A). None of the immunocompetent hosts died over this time period and upon sacrifice did not have enlarged spleens or livers (FIG. 5A). Southern blotting analysis of IgH rearrangements on the genomic DNA isolated from both enlarged spleen and liver from the immunodeficient recipients revealed the same or a subset of clonal rearrangements found in the liver donor cells (FIG. 5B). These finding demonstrate that, as in germline CLT mice, clonally transformed B cell clones grow out in both CLT and CLT; AID−/− RDBC mice as demonstrated by their transfer to immunodeficient hosts. Also, like LMP1+ B cell lymphomas from germline CLT mice, LMP1+ B cell tumors from CLT and CLT; AID−/− RDBC mice do not appear to be transferable into immunocompetent hosts.

CLT mice provide an extremely attractive model for studies of EBV-related pathologies and for studies of immune surveillance and approaches to cancer immunotherapy (8,9,25,26). Extension of this model for such experiments often would involve introduction of additional inactivating or activating mutations of other genes into the CLT background. Because of the difficulty of doing such experiments by germline breeding due to the great genetic complexity of the model, the inventors have developed and now documented the effectiveness of a rapid RDBC based model that utilizes CLT ES cells. The CLT RDBC chimeras obtained develop the same types of PTLD and B-cell lymphomas, with very similar kinetics to the germline CLT mice. The RDBC approach enables very efficient generation of large cohorts of CLT mice for analysis. Furthermore, the inventors proved the feasibility and efficiency of the approach for studies of other genes of interest in the CLT RDBC model by knocking out both copies of the AID gene in CLT ES cells via a CRISPR/Cas9 approach and then using the resultant AID−/− CLT ES cells for RDBC.

In the development of this new approach to generate CLT mouse tumor models, the inventors have also made several new findings regarding the CLT model. First, our studies show that despite the expansion of LMP1+ B cells in the spleen, there are essentially none in the peripheral blood of CLT RDBC chimeras. Without wishing to be bound by theory, one possibility would be that this phenomenon is related to known effects of CD40 or LMP1 activation on B cell mobility/homing and molecules related to that process (4,5,27,28). Most B cells tumors that arise in CLT mice express AID, indicating a possible role of AID expression in the tumorigenesis process (8,10,11). However, these current studies show that LMP1+ B cell expansions and tumors develop in AID−/− CLT model essentially identically to their development in the CLT model. In addition, these experiments show similar characteristics of CLT and CLT, AID−/− tumors following transplant immunodeficient and immuno-competent hosts. The CLT RDBC mouse model will greatly facilitate such future studies.

Going forward this ES cell-based B cell tumor model approach will allow ready knock-out or enforced expression of genes of interest in CLT ES cells to test their functions in the CLT mouse tumor model much more rapidly and efficiently than it would be possible with the germline CLT model. Thus, the CLT RDBC chimera B cell proliferation and lymphoma model will greatly facilitate future analyses of genetic alterations that cooperate with LMP1 to promote lymphomagenesis; genetic mechanisms that contribute to immune recognition of LMP1 driven lymphomas; and genetic mechanisms that contribute to immune escape of LMP1 driven lymphomas; while also potentially providing a model to develop immunotherapies that promote destruction of LMP1 immune escape variants. Finally, beyond the CLT B cell lymphoma model, the general ES cell-based blastocyst complementation chimera approach to tumor modeling can also be applied to other mouse tumor models that involve complex genetic modifications.

### REFERENCES

1. Kutok J L, et al. “Spectrum of Epstein-Barr virus-associated diseases.” Annual review of pathology 2006; 1:375-404.
2. Hislop A D, et al. “Cellular responses to viral infection in humans: lessons from Epstein-Barr virus.” Annual review of immunology 2007; 25:587-617.
3. Kaye K M, et al. “Epstein-Barr virus latent membrane protein 1 is essential for B-lymphocyte growth transformation.” Proceedings of the National Academy of Sciences of the United States of America 1993; 90(19):9150-4.
4. Uchida J, et al. “Mimicry of CD40 signals by Epstein-Barr virus LMP1 in B lymphocyte responses.” Science 1999; 286(5438):300-3.
5. Thorley-Lawson D A. “Epstein-Barr virus: exploiting the immune system.” Nature reviews Immunology 2001; 1(1):75-82.
6. Soni V, et al. “LMP1 TRAFficking activates growth and survival pathways.” Advances in experimental medicine and biology 2007; 597: 173-87.
7. Wang D, et al. “An EBV membrane protein expressed in immortalized lymphocytes transforms established rodent cells.” Cell 1985; 43(3 Pt 2):831-40.
8. Zhang B, et al. “Immune surveillance and therapy of lymphomas driven by Epstein-Barr virus protein LMP1 in a mouse model.” Cell 2012; 148(4):739-51.
9. Yasuda T, et al. “Studying Epstein-Barr virus pathologies and immune surveillance by reconstructing EBV infection in mice.” Cold Spring Harbor symposia on quantitative biology 2013; 78:259-63.
10. He B, et al. “EBV-encoded latent membrane protein 1 cooperates with BAFF/BLyS and APRIL to induce T cell-independent Ig heavy chain class switching.” Journal of immunology 2003; 171(10):5215-24.
11. Kim J H, et al. Epstein-Barr virus latent membrane protein 1 increases genomic instability through Egr-1-mediated up-regulation of activation-induced cytidine deaminase in B-cell lymphoma.” Leukemia & lymphoma 2013; 54(9):2035-40.
12. Muramatsu M, et al. “Class switch recombination and hypermutation require activation-induced cytidine deaminase (AID), a potential RNA editing enzyme.” Cell 2000; 102(5):553-63.
13. Alt F W, et al. “Mechanisms of programmed DNA lesions and genomic instability in the immune system.” Cell 2013; 152(3):417-29.
14. Kuppers R, et al. “Mechanisms of chromosomal translocations in B cell lymphomas.” Oncogene 2001; 20(40):5580-94.
15. Pasqualucci L, et al. “Hypermutation of multiple proto-oncogenes in B-cell diffuse large-cell lymphomas.” Nature 2001; 412(6844):341-6.
16. Ramiro A R, et al. “AID is required for c-myc/IgH chromosome translocations in vivo.” Cell 2004; 118(4):431-8.
17. Chen J, et al. “RAG-2-deficient blastocyst complementation: an assay of gene function in lymphocyte development.” Proceedings of the National Academy of Sciences of the United States of America 1993; 90(10):4528-32.
18. Shinkai Y, et al. “RAG-2-deficient mice lack mature lymphocytes owing to inability to initiate V(D)J rearrangement.” Cell 1992; 68(5):855-67.
19. Abbondanzo S J, et al. “Derivation of embryonic stem cell lines.” Methods in enzymology 1993; 225:803-23.
20. Cong L, et al. “Multiplex genome engineering using CRISPR/Cas systems.” Science 2013; 339(6121):819-23.
21. Guo C, et al. “CTCF-binding elements mediate control of V(D)J recombination.” Nature 2011; 477(7365):424-30.
22. Tepsuporn S, et al. “Mechanisms that can promote peripheral B-cell lymphoma in ATM-deficient mice.” Cancer immunology research 2014; 2(9):857-66.
23. Chaudhuri J, et al. “Transcription-targeted DNA deamination by the AID antibody diversification enzyme.” Nature 2003; 422(6933):726-30.
24. Le Clorennec C, et al. “EBV latency III immortalization program sensitizes B cells to induction of CD95-mediated apoptosis via LMP1: role of NF-kappaB, STAT1, and p53.” Blood 2006; 107(5):2070-8.
25. Savoldo B, et al. “Treatment of solid organ transplant recipients with autologous Epstein Barr virus-specific cytotoxic T lymphocytes (CTLs).” Blood 2006; 108(9):2942-9.
26. Gottschalk S, et al. “Post-transplant lymphoproliferative disorders.” Annual review of medicine 2005; 56:29-44.
27. Rickinson A B, et al. “Epstein-Barr virus: Co-opting B-cell memory and migration.” Current biology: CB 2000; 10(3):R120-3.
28. Babcock G J, et al. “Epstein-barr virus-infected resting memory B cells, not proliferating lymphoblasts, accumulate in the peripheral blood of immunosuppressed patients. The Journal of experimental medicine 1999; 190(4):567-76.

### Example 2: A Medulloblastoma Mouse Model & Roles of Recurrent DNA Break Cluster Genes in Neural Development and Disease

Summary:

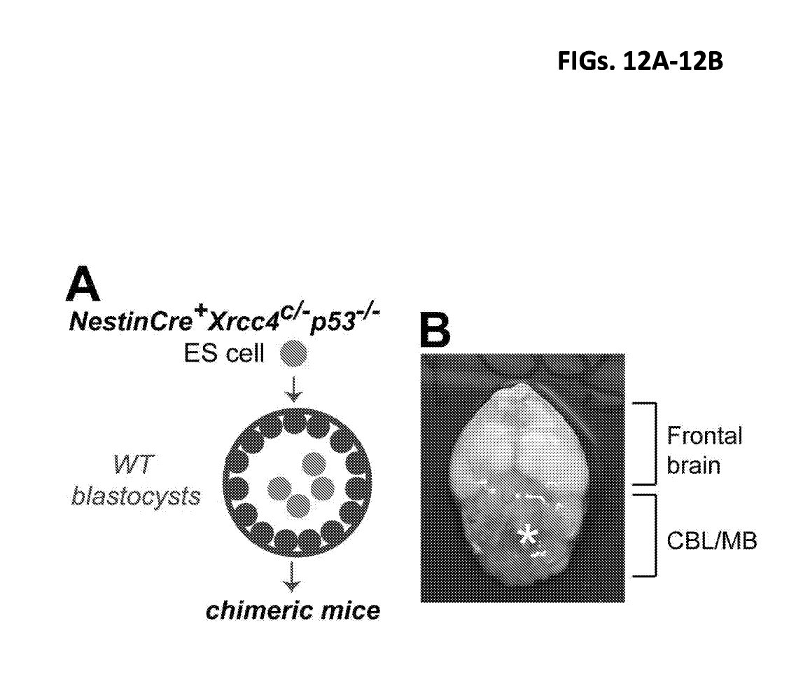

The inventors have shown that p53-deficient mice, in which Xrcc4 is inactivated in neural stem and progenitor cells via a Nestin-Cre based conditional knock-out approach, rapidly develop medulloblastomas (MBs) (Yan et al., 2006), the most common pediatric brain cancer and which causes the highest cancer-related mortality in children. XRCC4/p53-deficient MBs harbor clonal recurrent chromosomal rearrangements similar to those found in certain human MBs, including focal deletion of Ptch-1, amplification of N-myc or c-Myc, and recurrent chromosomal translocations (Yan et al., 2006). Extra-chromosomal N-myc amplification occurs in some of the tumors in association with double minutes (Yan et al., 2006), consistent with catastrophic rearrangement events that occur in the context of chromothripsis observed in human MBs (Rausch et al., 2012). To identify breakpoints of recurrent genomic alterations, one current goal is to perform whole genome sequencing of XRCC4/p53 deficient MBs. Because these studies require analyses of multiple individual tumors that harbor specific genomic aberrations (deletions, translocations, amplifications, etc.) of interest, the inventors have applied the blastocyst complementation approach to generate mouse MBs more efficiently. In one study, NestinCre/Xrcc4-conditional/p53-deficient (NestinCre⁺Xrcc4^{c/-}p53^{-/-}) ES cells were injected into wild-type blastocysts (FIG. 12A), and resulted in a first litter in which one mouse had contribution of NestinCre⁺, Xrcc4^{c/-} and p53-deleted alleles. Two siblings had no contribution. This chimeric mouse showed MB symptoms at 11-weeks of age and developed a MB in in the cerebellum, similarly to the conditional germline mice (FIG. 12B). The two siblings that did not have NestinCre⁺, Xrcc4^{c//}p53-deleted ES cell contribution were disease-free to at least ten-months of age. The blastocyst complementation method can allow efficient generation of large numbers of independent MBs within a 4-5 month period on an as needed basis.

Background:

DNA double strand breaks (DSBs) are generated in mammalian cells by cell intrinsic processes such as DNA replication, transcription, or oxidative stress (1, 2). Classical non-homologous end joining (C-NHEJ) is a major DSB repair pathway in somatic cells that repairs DSBs by joining their ends back together (3). Deficiency for evolutionarily conserved “core” C-NHEJ proteins including XRCC4 or DNA Ligase 4 (Lig4) leads to DSB persistence, which promotes joining to other DSBs that can generate chromosomal deletions, inversions, amplifications and translocations (3). In the absence of C-NHEJ, DSBs can still be joined, sometimes less efficiently, by alternative end-joining (A-EJ) pathways that may also contribute to translocations (4, 5). Previous studies have shown that C-NHEJ is required for immune and nervous system development. Thus, germline inactivation of Xrcc4 or Lig4 in mice blocks lymphocyte development by impairing assembly of antigen receptor variable region exons by V(D)J recombination and also severely impairs neural development by leading to widespread post-mitotic apoptosis of newly developed neurons (6-8). Deficiency for the p53 checkpoint protein rescues the neuronal loss phenotypes of XRCC4- or Lig4-deficient mice (9, 10). However, these double-deficient mice routinely die from progenitor B cell lymphomas with oncogenic clonal translocations and amplifications mediated by aberrant V(D)J recombination (11-13) and also develop medulloblastomas (MBs) in situ (12, 14). In the latter context, previous work from the inventors also showed that neural stem/progenitor cell (NSPC)-specific inactivation of Xrcc4 in p53-deficient mice leads to MBs that harbor recurrent genomic rearrangements (4) related to those seen in some subsets of human MBs (15, 16).

Brain cells, including the post-mitotic neurons, frequently contain somatic genomic variations, including deletions and rearrangements, which in some cases are linked to retrotransposon activities (17-19). Single-cell sequencing of 110 postmortem human frontal cortex neurons revealed that up to 41% of the neurons had Mb-scale de novo copy number variations (CNVs), most of which were deletions (17). Such somatic changes have been speculated to generate neuronal diversity (19, 20). Without wishing to be bound by theory, genomic aberrations in NSPCs might be transmitted to daughter cells and, thereby, contribute to genomic mosaicism in individual neurons or glial cells, where they could influence aspects of normal or abnormal brain function (18). A better understanding of potential impacts of such genomic alterations in neural cells has long awaited elucidation of underlying mechanisms (18, 19).

A previous study by the inventors has provided one such potential mechanism by identifying certain genes that undergo recurrent DSBs in neural stem and progenitor cells (NSPCs) (21). The inventors have developed and perfected an unbiased high-throughput, genome-wide, translocation sequencing (HTGTS) approach to map genome-wide DSBs at nucleotide resolution. HTGTS is based on the ability of genome-wide “prey” DSBs to translocate to recurrent ectopic (e.g. generated by designer nucleases) or endogenous (e.g. V(D)J or class switch recombination DSBs in immunoglobulin genes) “bait” DSBs at a specific chromosomal location (13, 22-24). Notably, for both mouse and human cells, recurrent endogenous prey DSBs or DSB clusters across the genome generally are captured by bait DSBs regardless of the chromosomal location of the bait (24), a property facilitated by cellular heterogeneity in 3-D genome organization (3, 24, 25). Thus, this phenomenon allows recurrent DSBs at a wide variety of genomic locations to be reliably identified by HTGTS baits on different chromosomes (24). To be joined, two DSBs also must lie in close spatial proximity, a property that greatly enhances the joining of separate DSBs in cis along a given chromosome (3, 24, 25). Within a cis chromosome, translocation frequency is further enhanced between sequences within topologically associated domains (TADs) or loops due to increased interaction or other processes (3, 13, 25). Together, these properties make HTGTS exceptionally sensitive for mapping, at nucleotide resolution, genome-wide recurrent classes of DSBs based on their translocation to bait DSBs.

Recently, HTGTS has been applied to map DSBs in NSPCs. These studies revealed 27 recurrent DSB clusters (“RDCs”) that all fall across the entire body of long neural genes that each lies within a single replication domain that corresponds to a topologically-associating domain (TAD) (21). Nearly all NSPC RDC genes encode surface proteins involved in synaptogenesis and related process and variations in most RDC genes have been implicated in neuropsychiatric disorders, including schizophrenia, bipolar disorder, and autism (21). In addition, a substantial fraction of these RDC genes are deleted or amplified in certain human MBs or other tumor types including glioblastomas and prostate cancer (21). The majority of the RDC-genes become apparent only after subjecting NSPCs to mild replication stress by treatment with aphidicolin (21). However, based on several lines of evidence it is thought that most or all RDC genes undergo frequent internal breakage without such treatment, due to endogenous stress, but that aphidicolin raises levels to those readily detectable by HTGTS (21). Indeed, a number of RDCs are located within genes that map to “common fragile sites” found cytogenetically in several mouse and human cell types (e.g. lymphocytes and fibroblasts) and which may contribute to genomic instability in cancer. In this regard, it is suggested that RDC DSBs may provide a nucleotide-resolution view of such fragile genomic sites (21).

HTGTS only finds the subset of DSBs in an RDC-gene that translocate. In this regard, these studies suggest that a much higher frequency of DSBs actually occur in RDC genes, but that they are repaired within the RDC gene by re-joining or joining to other DSBs within it due to their location within a single TAD (21). Other work from the inventors has shown that B lymphocytes exploit the tendency of DSBs within a TAD or chromosomal loop domain to join to other DSBs within that domain in the context of V(D)J recombination and IgH class switch recombination (CSR) (3, 13, 23, 26). IgH CSR of Ig genes involves joining of intronic DSBs to delete one set of constant region exons and replace it with an adjacent set to that lie downstream (3). By analogy, it is hypothesized that various NSPC RDC genes that harbor many small exons embedded within long introns might similarly employ joining of frequent DSBs within them to produce different variants of encoded products, thereby contributing to neural cell diversity. Likewise, some such recombination events might contribute to disease-associated mutations. As a number of such RDC-genes encode a large set of variant proteins that express different combinations of exons due to differential RNA splicing, it is hypothesized that the RDC-based recombination process might “hard-wire” the expression of such variants. Such a mechanism could contribute to diversifying the NSPCs population, and therefore generate genomic mosiacism of brain cells and diversification of neural cell function. In this regard, the human counterparts of 9 of the 27 NSPC RDCgenes have been found in relatively focal CNVs found in individual human frontal cortex neurons (17), consistent with the possibility that frequent NSPC RDC DSBs may contribute to genomic variations that are carried at significant levels through development into mature neurons.

In order to determine the roles of recurrent double strand break clusters in NSPCs in neuronal diversification and disease, the HTGTS assay can be adapted to use DSBs within individual endogenous RDC genes as HTGTS baits to identify joining patterns of their DSBs and, thereby, identify the potential variant forms of their encoded proteins that could be expressed. To assess potential significance of HTGTS findings, deep RNA-seq analyses of RDC genes in NSPCs and mature neurons can be performed, to assess expression of potential new transcripts implicated by HTGTS. As RDC gene DSBs are greatly enhanced by mild replication stress induced by aphidicolin, RNA-seq using cells treated with or without aphidicolin can be performed to assess linkage of variant transcript production with generation of RDC DSBs.

In some embodiments, the methods described herein are used to identify DSBs that contribute to recurrent genomic variations found in MBs. For example, the MB model described herein can be modified to rapidly derive large numbers of these tumors for use to deep sequence multiple independent MBs to identify the breakpoints of the recurrent genomic alterations. These breakpoints can be compared with RDCs or other DSB clusters found in NSPCs and also those found in a search for cultured cerebellar granule neuron progenitors (CGNPs) from which MBs arise. The CGNP studies can be done by the same general approaches previously used to identify RDCs in NSPCs, including use of both WT and DSB repair deficient cells. Such analyses can be enhanced by employing introduced HTGTS bait DSBs in the region of genomic MB alterations identified by deep sequencing of the tumors to facilitate identification of potential underlying DSBs via 3D proximity effects.

Methods & Results:

In one embodiment, HTGTS is employed to identify joining patterns of the independent endogenous DSBs within RDC-genes and thereby to identify potential rearrangements within them and any resulting variant forms of the proteins encoded by such rearrangements. Initial studies can focus on a subset of RDC genes including Lsamp which has 9 exons (27, 28) and Npas3 which has 12 exons (29, 30). Both Lsamp and Npas3 are known to express more than one encoded product (28, 30) and preliminary RNA-Seq analysis of cerebral cortex from adult mice in the context of unrelated studies in the lab on effects of Sirtuin 6 deficiency and gene expression in the brain indicates at least 6 detectably expressed isoforms of Lsamp and 14 of Npas3 (data not shown). Initial studies can foucs on these two RDC genes because they are the most fragile of those that we have identified, a property that will aid in establishing requisite technologies. In further studies, the inventors can focus on other RDC genes starting with Neurexin (Nrxn)-1 and Nrxn-3. The analysis of Nrxn-1 and Nrxn-3 is of particular interest since these two RDC genes each have more than 20 exons embedded across their long gene bodies. Moreover, the Nrxn genes have the capacity to encode large numbers of alternatively spliced transcript variants: differential expression of such variants can contribute to mosaic expression repertoires of neurons in different regions of the mouse brain (31). Thus, it will be of interest to determine if joining of independent endogenous DSBs within Nrxn-1 and Nrxn-3, as well as in other RDC genes, could contribute to generating expression of diverse transcripts in NSPCs and, if so, whether any such variants could contribute to gene expression repertoire diversity in mature neurons.

Use of Endogenous NSPC RDC DSBs to Elucidate Intra-Genic Recombination Events.

The inventors can adapt their HTGTS assay to use endogenous DSBs within a given RDC-gene as HTGTS bait. The approach of employing endogenous DSBs as bait is analogous to previous methods used to elucidate joining of separate sets of IgH CSR breaks (23) within a TAD in the IgH locus and the joining of V(D)J recombination-associated DSBs within various TADs across the mouse genome (13). The use of endogenous RDC DSBs in a given RDC gene as HTGTS intra-genic bait permits sensitive identification of DSBs within the RDC gene; because most of these genes are thought to lie within a single TAD (21); and DSB joining of DSBs within the same TAD is greatly enhanced due to proximity effects (3, 25). Moreover, this approach permits one to identify joining events between DSBs within a given RDC gene that lead to intra-genic deletions and inversions (23) that might lead to variant forms of expressed transcripts. All of these HTGTS experiments can be performed on both WT and XRCC4/p53 deficient cells, with the latter C-NHEJ deficient background used to potentially enhance DSB recovery (21). The experiments also can be done both in control NSPCs and NSPCs treated with aphidicolin (21), both to increase DSB levels in the RDC genes and also to provide NSPC populations that harbor cells that may have increased levels of any variant RNA transcripts associated with joining between the increased DSBs. In subsequent experiments, similar analyses of Npas3, Nrxn-1, and Nrxn-3 can be performed. The assay can be readily extended to additional RDC genes.

Preliminary Intra-Genic HTGTS Data for Lsamp Endogenous DSBs.

To establish this intra-genic HTGTS approach, HTGTS primers were first designed that target a region within intron 2 of Lsamp that is enriched in RDC DSBs (FIG. 11). The intra-genic HTGTS assay for Lsamp can be further optimized by testing primers at various locations across the gene to compare joining patterns and allowing the optimization of junctional recovery levels. First, an endogenous HTGTS primer that lies within intron 2 of Lsamp, the region shown to have a high density of DSBs based on HTGTS studies using ectopically-introduced HTGTS bait DSBs outside of Lsamp on Chr16 or ectopic bait DSBs on other chromosomes (21), can be employed. For the initial studies, immediately available ATM-deficient NSPCs were employed, in which the Lsamp gene contains an RDC even in the absence of aphidicolin treatment (21). In these preliminary experiments 28 junctions were detected between the endogenous Lsamp DSBs in intron 2 and other DSBs within the Lsamp gene body (FIG. 11A). The joining of these separate endogenous DSBs in Lsamp recovered thus far mostly represented kilobase-scale deletions within intron 2 (n=21; FIG. 11B). However, other types of intragenic recombination events were observed, including those representing deletions of Exon 2 (n=1), deletion of Exon 3 (n=1), deletion of Exon 3 and 4 (n=1) and intragenic inversions (n=4) (FIG. 11B). As a control, the same Lsamp intron 2 primer was used to generate HTGTS libraries from DNA of in vitro CSR-activated B lymphocytes, in which the Lsamp gene does not contain recurrent DSBs (21), and did not detect any intragenic DSB junctions within Lsamp (data not shown). To extend these preliminary studies, the HTGTS studies can be scaled up to obtain greater numbers of DSBs and further optimize recovery of junctions across the gene by employing bait primers located in different regions. In addition the studies can be extended to both WT and XRCC4/p53-deficient backgrounds and in the presence or absence of aphidicolin treatment which, based on recent studies (21), should increase recovery of intra-genic DSB recombination events.

RNA Seq.:

To assess potential linkage of recombination/deletion events between different DSBs identified within a given RDC gene with ability to lead to variant transcripts, deep RNA-seq analyses of NSPC transcripts from WT and XRCC4/p53 deficient NSPC cells can be performed, both with and without aphidicolin treatment. The RNA-seq method (32) is well established and has previously been applied to compare expression profiles of mature wild-type and mutant neurons in the context of unrelated studies as mentioned above. The RNA-Seq experiments permit one of skill in the art to assess correlations between types or levels of expressed alternative transcripts and observed rearrangement patterns of RDC genes. In particular, one can assay for differences in type and levels of detected alternative transcripts of RDC genes in NSPCs that are either untreated or treated with aphidicolin. Any reproducible changes in transcript variants or levels of particular variants would provide strong evidence for a potential link between recombination between of DSBs in RDC genes and ability to generate variant products. To further access whether such potential genomic alterations can be carried developmentally into mature neurons, one can also perform RNA-seq analyses of neurons differentiated from untreated and aphidicolin-treated NSPCs and also on mature neurons from the WT and XRCC4/p53-deficient NSPCs. Deep RNA-Seq analyses from wild-type cerebral cortex can be analyzed in depth for RDC gene variant isoforms. These studies can provide information for further assessing whether changes of alternative transcript repertoires of RDC genes in NSPCs have the potential to impact on functions of mature neurons.

In another embodiment, methods provided herein can be used to identify DSBs that contribute to recurrent genomic variations in MBs. It has been shown that p53-deficient mice, in which Xrcc4 is inactivated in NSPCs via a Nestin-Cre based conditional knock-out approach rapidly develop MBs (4). XRCC4/p53-deficient MBs harbor clonal recurrent chromosomal rearrangements similar to those found in certain human MBs, including focal deletion of Ptch-1, amplification of N-myc or c-Myc, and recurrent chromosomal translocations (4). Extra-chromosomal N-myc amplification occurs in some of the tumors in association with double minutes (4), consistent with catastrophic rearrangement events that occur in the context of chromothripsis (16). One can perform whole genome deep sequencing of XRCC4/p53 deficient MBs to identify breakpoints of recurrent genomic alterations. For these studies, a rapid method was devised to generate mice that develop these tumors, which will allow an increase in sample size. In parallel, the location of breakpoints found in MBs is compared to the location of RDCs in NSPCs. In addition, the HTGTS analyses are extended to cultured cerebellar granule neuron progenitors (CGNPs), which contain the precursor population that gives rise to MBs.

Identifying Breakpoints of Recurrent Genomic Alterations in Mouse MBs.

The inventors have employed whole genome sequencing of MBs from their mouse model. Because these studies may require analysis of multiple individual tumors that harbor particular genomic aberrations (deletions, translocations, amplifications, etc.) of interest, the inventors developed a method to generate mouse MBs more efficiently. This approach involves generating chimeric mice derived from injection of NestinCre/Xrcc4− conditional/p53-deficient ES cells into wild-type blastocysts (FIG. 12A). Preliminary studies show that chimeras generated by this approach develop MBs similarly to the more cumbersome conditional germline model (FIG. 12B). The new approach allows generation of large numbers of new MBs within a 4-5 month period on an as needed basis. Once tumors are generated, they are characterized cytogenetically for chromosomal aberrations and by Southern blotting for recurrent amplifications as described (4). MB genomes can be sequenced and analyzed, which will provide the locations of breakpoints and other genomic aberrations.

Identification of Recurrent DSBs that can Contribute to Genomic Aberrations in Mouse MBs.

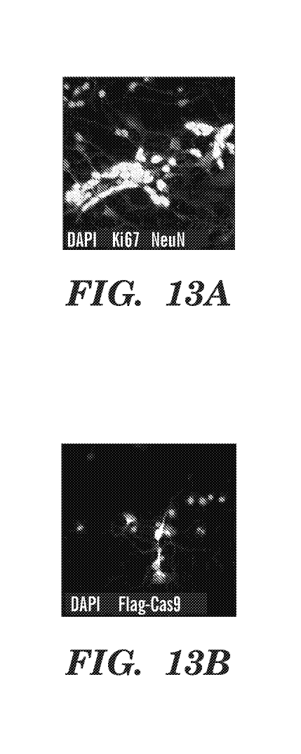

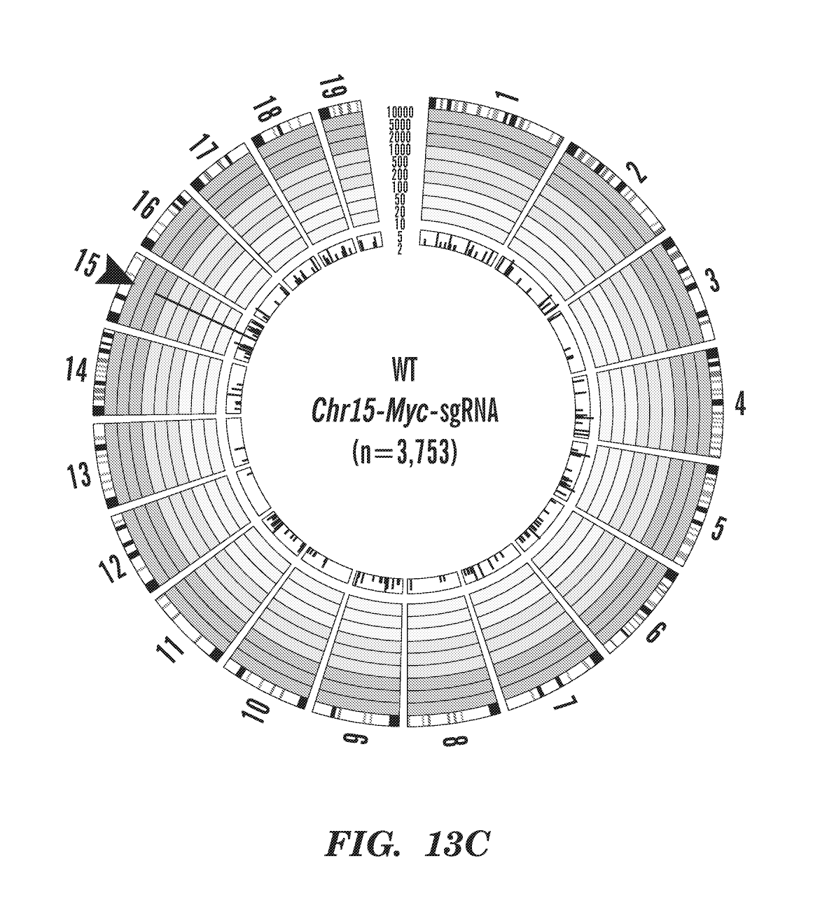

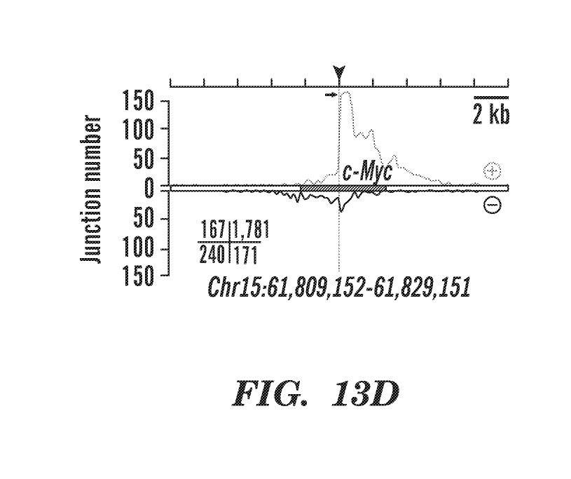

Some of the 27 robust NSPC RDCs occur near genes associated with lesions found in human MB (21). Breakpoints of genomic aberrations found by whole genome sequencing of MBs (see above) can be compared with locations of these 27 and also to those of several hundred additional lower level candidate RDCs described previously (21). Any of the latter closely linked to breakpoints of MB chromosomal lesions can be confirmed with additional HTGTS baits and further characterized as described (21). The HTGTS studies can be extended to wild-type and XRCC4/p53-deficient cultured primary mouse CGNPs, which are expected to contain the direct precursor cells of MB. The HTGTS bait DSB induction method has been optimized by using a lentiviral-based expression system to deliver Cas9/sgRNA. For this purpose, cerebellar granule cell cultures were prepared in the presence of sonic hedgehog (SHH), which facilitates their proliferation in vitro (36, 37). Approximately 20% of the cells in these cultures were actively proliferating at day 4.5 based on Ki67 expression (FIG. 13A). The majority of these proliferating cells lacked expression of a mature neuron marker (NeuN) (FIG. 13A), indicating they could be CGNPs. Cultures were transduced at day 1 with lentivirus expressing the Flag-Cas9/Chr15-Myc-sgRNA extensively characterized in prior studies (21, 38).

Approximately 50% of the cells expressed Flag-Cas9 (FIG. 3B) at day 4.5. An HTGTS library was generated from day 4.5 cultures, which revealed that Cas9/sgRNA transduction efficiency was sufficient to generate a large peak of DSBs at the bait site that mainly consist of rejoining resected ends of the bait DSBs (FIG. 13C-13D). In addition, an expected fraction of the bait DSBs translocate to other DSBs genome wide (FIG. 13C). This general pattern is consistent with what has been found for successful HTGTS studies of other mouse B lymphocytes and NSPCs (21, 22), and human 293T cells (24). Thus, these preliminary studies confirm that one can successfully generate HTGTS libraries from cerebellar granule cell cultures. For ongoing and future studies, current HTGTS libraries can be scaled up to achieve sufficient numbers of junctions to assay for recurrent classes of DSBs, including RDCs, wide-spread low-level DSBs (revealed by increased junction density along the break-site chromosome) and transcription startsite associated DSBs. These analyses can be performed as described in recent publications on NSPC HTGTS studies (21, 38). Finally, the inventors have obtained MATH1-GFP mice, which express GFP in CGNPs (39); to enrich CGNPs for HTGTS experiments, to explore sorting GFP+ cells from cerebellum before culturing with SHH. One can extend both the NSPC and proposed CGNP HTGTS analyses by introducing HTGTS bait DSBs in the region of any potential recurrent genomic MB alterations identified using deep sequencing of the MBs. This approach, analogous to what was outlined for employing endogenous RDC breaks as HTGTS baits above, can facilitate identification of recurrent DSBs that contribute to genomic variations in MB tumors via 3D proximity effects.

### REFERENCES

1. Aguilera A & Garcia-Muse T (2013) Causes of genome instability. Annual review of genetics 47:1-32.
2. Kim N & Jinks-Robertson S (2012) Transcription as a source of genome instability. Nature reviews.
Genetics 13(3):204-214.
3. Alt F W, Zhang Y, Meng F L, Guo C, & Schwer B (2013) Mechanisms of programmed DNA lesions and genomic instability in the immune system. Cell 152(3):417-429.
4. Yan C T, et al. (2006) XRCC4 suppresses medulloblastomas with recurrent translocations in p53-deficient mice. Proc Natl Acad Sci USA 103(19):7378-7383.
5. Boboila C, Alt F W, & Schwer B (2012) Classical and alternative end-joining pathways for repair of lymphocyte-specific and general DNA double-strand breaks. Adv Immunol 116(1-49).
6. Barnes D E, Stamp G, Rosewell I, Denzel A, & Lindahl T (1998) Targeted disruption of the gene encoding DNA ligase IV leads to lethality in embryonic mice. Current biology:CB 8(25): 1395-1398.
7. Frank K M, et al. (1998) Late embryonic lethality and impaired V(D)J recombination in mice lacking DNA ligase IV. Nature 396(6707):173-177.
8. Gao Y, et al. (1998) A critical role for DNA end-joining proteins in both lymphogenesis and neurogenesis. Cell 95(7):891-902.
9. Frank K M, et al. (2000) DNA ligase IV deficiency in mice leads to defective neurogenesis and embryonic lethality via the p53 pathway. Mol Cell 5(6):993-1002.
10. Gao Y, et al. (2000) Interplay of p53 and DNA-repair protein XRCC4 in tumorigenesis, genomic stability and development. Nature 404(6780):897-900.
11. Difilippantonio M J, et al. (2002) Evidence for replicative repair of DNA double-strand breaks leading to oncogenic translocation and gene amplification. The Journal of experimental medicine 196(4):469-480.
12. Zhu C, et al. (2002) Unrepaired DNA breaks in p53-deficient cells lead to oncogenic gene amplification subsequent to translocations. Cell 109(7):811-821.
13. Hu J, et al. (2015) Chromosomal loop domains direct the recombination of antigen receptor genes. Cell 163(4):947-959.
14. Lee Y & McKinnon P J (2002) DNA ligase IV suppresses medulloblastoma formation. Cancer research 62(22):6395-6399.
15. Northcott P A, et al. (2012) Subgroup-specific structural variation across 1,000 medulloblastoma genomes. Nature 488(7409):49-56.
16. Rausch T, et al. (2012) Genome sequencing of pediatric medulloblastoma links catastrophic DNA rearrangements with TP53 mutations. Cell 148(1-2):59-71.
17. McConnell M J, et al. (2013) Mosaic copy number variation in human neurons. Science 342(6158):632-637.
18. Poduri A, Evrony G D, Cai X, & Walsh C A (2013) Somatic mutation, genomic variation, and neurological disease. Science 341(6141):1237758.
19. Erwin J A, Marchetto M C, & Gage F H (2014) Mobile DNA elements in the generation of diversity and complexity in the brain. Nature reviews. Neuroscience 15(8):497-506.
20. Muotri A R, et al. (2005) Somatic mosaicism in neuronal precursor cells mediated by L1 retrotransposition. Nature 435(7044):903-910.
21. Wei P C, Chang A N, Kao J, Du Z, Meyers R M, Alt F W, Schwer B (2016) Long neural genes harbor recurrent DNA break clusters in neural stem/progenitor cells. Cell In Press.
22. Chiarle R, et al. (2011) Genome-wide translocation sequencing reveals mechanisms of chromosome breaks and rearrangements in B cells. Cell 147(1):107-119.
23. Dong J, et al. (2015) Orientation-specific joining of AID-initiated DNA breaks promotes antibody class switching. Nature 525(7567):134-139.
24. Frock R L, et al. (2015) Genome-wide detection of DNA double-stranded breaks induced by engineered nucleases. Nature biotechnology 33(2): 179-186.
25. Zhang Y, et al. (2012) Spatial organization of the mouse genome and its role in recurrent chromosomal translocations. Cell 148(5):908-921.
26. Zarrin A A, et al. (2007) Antibody class switching mediated by yeast endonuclease-generated DNA breaks. Science 315(5810):377-381.
27. Pimenta A F & Levitt P (2004) Characterization of the genomic structure of the mouse limbic system associated membrane protein (Lsamp) gene. Genomics 83(5):790-801.
28. Rosenbloom K R, et al. (2015) The UCSC Genome Browser database: 2015 update.
Nucleic acids research 43(Database issue):D670-681.
29. Brunskill E W, Witte D P, Shreiner A B, & Potter S S (1999) Characterization of npas3, a novel basic helixloop-helix PAS gene expressed in the developing mouse nervous system. Mechanisms of development 88(2):237-241.
30. McGarvey K M, et al. (2015) Mouse genome annotation by the RefSeq project. Mammalian genome: official journal of the International Mammalian Genome Society 26(9-10):379-390.
31. Schreiner D, et al. (2014) Targeted combinatorial alternative splicing generates brain region-specific repertoires of neurexins. Neuron 84(2):386-398.
32. Trapnell C, et al. (2012) Differential gene and transcript expression analysis of RNA-seq experiments with TopHat and Cufflinks. Nature protocols 7(3):562-578.
33. Ba Z, et al. (2015) A Rapid Embryonic Stem Cell-Based Mouse Model for B-cell Lymphomas Driven by Epstein-Barr Virus Protein LMP1. Cancer immunology research 3(6):641-649.
34. Jones D T, et al. (2012) Dissecting the genomic complexity underlying medulloblastoma. Nature 488(7409):100-105.
35. Hovestadt V, et al. (2014) Decoding the regulatory landscape of medulloblastoma using DNA methylation sequencing. Nature 510(7506):537-541.
36. Wechsler-Reya R J & Scott M P (1999) Control of neuronal precursor proliferation in the cerebellum by Sonic Hedgehog. Neuron 22(1): 103-114.
37. Lee H Y, Greene L A, Mason C A, & Manzini M C (2009) Isolation and culture of post-natal mouse cerebellar granule neuron progenitor cells and neurons. Journal of visualized experiments: JoVE (23).
38. Schwer B, Wei P C, Chang A N, Kao J, Du Z, Meyers R M, Alt F W (2016) Transcription-associated processes cause DNA double-strand breaks and translocations in neural stem/progenitor cells. Proc Natl Acad Sci USA In Press.
39. Rose M F, et al. (2009) Math1 is essential for the development of hindbrain neurons critical for perinatal breathing. Neuron 64(3):341-354.
40. Rao S S, et al. (2014) A 3D map of the human genome at kilobase resolution reveals principles of chromatin looping. Cell 159(7): 1665-1680.
41. Platt R J, et al. (2014) CRISPR-Cas9 knockin mice for genome editing and cancer modeling. Cell 159(2):440-455.

1. A method for generating a transgenic mouse lymphoma model, the method comprising:
(a) introducing an embryonic stem (ES) cell or induced pluripotent stem (iPS) cell comprising a modified genome to a RAG-2-deficient blastocyst to produce a chimeric blastocyst, and
(b) implanting the chimeric blastocyst into a female mouse for gestation, thereby generating a transgenic mouse lymphoma model.

2. The method of claim 1, wherein the ES cell or iPS cell is derived from a tumor model mouse.

3.-4. (canceled)

5. The method of claim 1, wherein the modified genome comprises addition of an activated oncogene or a latent viral gene.

6. The method of claim 1, wherein the transgenic mouse comprises (i) lymphocytes having the genotype of the ES or iPS cell or (ii) B and/or T cells having the genotype of the ES or iPS cell.

7.-10. (canceled)

11. The method of claim 1, wherein the method provides transgenic mice faster than conventional back-crossing methods.

12. A transgenic mouse lymphoma model made by the method of claim 1.

13. A RAG-2 deficient transgenic mouse lymphoma model comprising: chimeric lymphocytes having a modified genome, wherein the genotype of the lymphoma is that of the chimeric lymphocytes.

14. The transgenic mouse of claim 13, wherein at least 50% of the lymphocytes in the transgenic mouse are chimeric lymphocytes.

15. (canceled)

16. The transgenic mouse of claim 13, wherein the modified genome comprises a latent viral gene or an oncogene.

17.-18. (canceled)

19. The transgenic mouse of claim 13, further comprising at least one symptom of a B cell expansion or a B cell lymphoma.

20. The transgenic mouse of claim 19, wherein the at least one symptom is selected from the group consisting of: splenomegaly, splenic tumor nodules, hepatomegaly, and hepatic tumor nodules.

21. (canceled)

22. The transgenic mouse of claim 13, wherein the chimeric lymphocytes are derived from a tumor mouse model.

23.-25. (canceled)

26. A screening assay for identifying an anti-cancer agent for the treatment of lymphoma, the assay comprising:
(a) determining the extent of B cell expansion or lymphoma in a RAG-2 deficient transgenic mouse,
wherein the RAG-2 deficient transgenic mouse comprises chimeric lymphocytes having a modified genome and further comprises a lymphoma having a genotype of the chimeric lymphocytes,
(b) administering a candidate anti-cancer agent to the RAG-2 deficient mouse, and
(c) comparing the extent of B cell expansion or lymphoma in the treated RAG-2 deficient mouse, wherein a decrease in the extent of B cell expansion or lymphoma identifies the candidate as an anti-cancer agent for treatment of lymphoma.

27. The assay of claim 26, wherein the extent of B cell expansion or lymphoma is determined by measuring spleen size.

28. (canceled)

29. A method for generating a complex mouse tumor model, the method comprising:
(a) introducing an embryonic stem (ES) cell or induced pluripotent stem (iPS) cell derived from a complex mouse tumor model and having a modified genome to a donor blastocyst to produce a chimeric blastocyst, and
(b) implanting the chimeric blastocyst into a female mouse for gestation, thereby generating a complex mouse tumor model.

30. The method of claim 29, wherein the tumor model mouse is homozygous recessive for one or more tumor-related genes.

31. (canceled)

32. The method of claim 29, wherein the modified genome comprises addition of an activated oncogene.

33. The method of claim 29, wherein the method provides transgenic mice faster than conventional back-crossing methods.

34. A complex mouse tumor model made by the method of claim 29.

35.-41. (canceled)

42. The method of claim 29, wherein the complex tumor mouse model is a medulloblastoma mouse model.


SEQUENCE LISTING (8 sequences)

SEQ ID NO: 1 - DNA, 23 bp, Artificial Sequence
  Description of Artificial Sequence Synthetic oligonucleotide
gtaggtctcatgccgtcccttgg

SEQ ID NO: 2 - DNA, 23 bp, Artificial Sequence
  Description of Artificial Sequence Synthetic oligonucleotide
gccgaagtccagtgagcaggagg

SEQ ID NO: 3 - DNA, 23 bp, Artificial Sequence
  Description of Artificial Sequence Synthetic oligonucleotide
ggattttgaaagcaacctcctgg

SEQ ID NO: 4 - DNA, 23 bp, Artificial Sequence
  Description of Artificial Sequence Synthetic oligonucleotide
gcgagatgcatttcgtatgttgg

SEQ ID NO: 5 - DNA, 20 bp, Artificial Sequence
  Description of Artificial Sequence Synthetic primer
gggccaagggacggcatgag

SEQ ID NO: 6 - DNA, 20 bp, Artificial Sequence
  Description of Artificial Sequence Synthetic primer
cccgggtccaggtcccagtc

SEQ ID NO: 7 - DNA, 19 bp, Artificial Sequence
  Description of Artificial Sequence Synthetic primer
gtcatgccgacccgcagtc

SEQ ID NO: 8 - DNA, 33 bp, Artificial Sequence
  Description of Artificial Sequence Synthetic primer
gtcctgtccataatcagtccatgaggaataaac